(19) United States
(12) Patent Application Publication
(10) Pub. No.: US 2025/0387475 A1
(43) Pub. Date: Dec. 25, 2025

(54) METHODS OF TREATING THE EFFECTS OF CYTOKINE STORMS

(71) Applicant: Sumant Singh Chugh, Chicago, IL (US)

(72) Inventor: Sumant Singh Chugh, Chicago, IL (US)

(73) Assignee: RUSH UNIVERSITY MEDICAL CENTER, Chicago, IL (US)

(21) Appl. No.: 18/704,783

(22) PCT Filed: Oct. 20, 2022
(86) PCT No.: PCT/US2022/047254

§ 371 Date: Apr. 25, 2024

Related U.S. Application Data
(60) Provisional application No. 63/272,402, filed on Oct. 27, 2021.

(51) Int. Cl. A61K 39/395; A61K 39/00; A61P 31/14; C07K 16/24

(52) U.S. Cl. CPC A61K 39/3955; A61P 31/14; C07K 16/241; C07K 16/244; C07K 16/246; C07K 16/247; C07K 16/248; C07K 16/249; A61K 2039/505; A61K 2039/507; C07K 2317/76

(57) ABSTRACT

The invention provides novel approaches to the prevention and treatment of the respiratory or non-respiratory infections and effects of mild to severe cytokine storms including multiorgan effects as well as the reduction in mortality. More particularly, the invention describes specific combinations or cytokines or soluble receptors that must be depleted to eliminate or reduce mortality as the result of severe viral cytokine storms.

### REFERENCE TO GOVERNMENT GRANTS

This invention was made with government support under R01 DK129522, R01 DK128203, R01 DK109713, and R01 DK111102 awarded by the National Institutes of Health. The government has certain rights in the invention.

### SEQUENCE LISTING

An electronic sequence listing (Chugh Dual Cytokine Inhibition.xml; size 32.0 KB; date of creation May 27, 2025) submitted herewith is incorporated by reference in its entirety.

### FIELD OF THE INVENTION

The general field of the present disclosure are novel approaches to the prevention and treatment of the effects of cytokine storms. The invention describes specific combinations or cytokines or soluble receptors that must be depleted to eliminate or reduce mortality as the result of severe viral cytokine storms.

### BACKGROUND

A striking feature of the COVID-19 pandemic is multisystem involvement including the respiratory tract, kidney, brain, liver, heart, gastro-intestinal tract, eyes and many other organs. See Huang et al., “Clinical features of patients infected with 2019 novel coronavirus in Wuhan, China,” (2020) Lancet 395: pp. 497-506; Wang et al., “Clinical features of 69 cases with coronavirus disease 2019 in Wuhan, China,” (2020) Clin Infect Dis. 71: pp. 769-777. The virus is not always detected in affected organs, and its presence or absence in cardiac autopsy studies did not appear to influence the extent of inflammatory cell infiltration. See Spudich et al., “Nervous system consequences of COVID-19,” (2022) Science 375: pp. 267-269; Topol, “COVID-19 can affect the heart,” (2020) Science 370: pp. 408-409; Lindner et a., “Association of cardiac infection with SARS-COV-2 in confirmed COVID-19 autopsy cases.” (2020) JAMA Cardiol. 5: pp. 1281-1285; Gupta, et al., “Extrapulmonary manifestations of COVID-19,” (2020) Nat Med. 26: pp. 1017-1032.

Viral infections trigger cytokine production as part of the innate and adaptive immune response. The inventors previously suspected that the extensive cytokine storm documented early in the pandemic may be involved in organ damage and developed novel evidence-based models of cytokine mediated end organ damage. See Huang et al. 2020. Of the three organs studied, the literature on cardiac involvement shows elevated cardiac Troponin I levels (that mimic an acute myocardial infarction), myocarditis, myocardial necrosis, pericarditis, arrythmias and heart failure4, 7. See Topo 2020; Inciardi et al., “Cardiac involvement in a patient with coronavirus disease 2019 (COVID-19),” (2020) JAMA Cardiol. 5: pp. 819-824. Evidence of liver injury include increased aminotransferase levels, hepatocyte injury, inflammation and steatosis8. See Herta et al., “COVID-19 and the liver-Lessons learned,” (2021) Liver Int. 41 Suppl 1: pp. 1-8. Kidney manifestations are very common in hospitalized COVID-19 patients, with nearly 40% developing proteinuria, and about one-third developing acute kidney injury (AKI). See Cheng et al., “Kidney disease is associated with in-hospital death of patients with COVID-19,” (2020) Kidney Int. 97: pp. 829-838; Hirsch et al., “Acute kidney injury in patients hospitalized with COVID-19,” (2020) Kidney Int. 98: pp. 209-218. Kidney biopsy studies in COVID-19 patients with severe proteinuria and/or kidney dysfunction have most commonly documented the collapsing variant of focal and segmental glomerulosclerosis (FSGS) and acute kidney injury. See Kudose et al., “Kidney Biopsy Findings in Patients with COVID-19,” (2020) J Am Soc Nephrol. 31: pp. 1959-1968; Nasr et al., “Kidney Biopsy Findings in Patients With COVID-19, Kidney Injury and Proteinuria,” (2021) Am J Kidney Dis. 77: pp. 465-468 (2021). Despite suspicion of viral particles in early autopsy studies, kidney biopsies from living patients did not note any viral particles. See also Bradley et al., “Histopathology and ultrastructural findings of fatal COVID-19 infections in Washington State: a case series,” (2020) Lancet 396: pp. 320-332.

The advantage of building a COVID-19 cytokine storm model around kidney disease is a potential mechanistic comparison with rare manifestations of a common cold cytokine storm, with which it shares some components. See Basnet et al., “Rhinoviruses and Their Receptors,” (2019) Chest 155: pp. 1018-1025; Wine et al., “Cytokine responses in the common cold and otitis media,” (2012) Curr Allergy Asthma Rep. 12: pp. 574-581; Nieters et al., “Cross-sectional study on cytokine polymorphisms, cytokine production after T-cell stimulation and clinical parameters in a random sample of a German population,” (2001) Hum Genet. 108: pp. 241-248; Noah et al., “Nasal cytokine production in viral acute upper respiratory infection of childhood,” (1995) J Infect Dis. 171: pp. 584-592; van Kempen et al., “An update on the pathophysiology of rhinovirus upper respiratory tract infections,” (1999) Rhinology 37: pp. 97-103; Whiteman et al., “IFN-gamma regulation of ICAM-1 receptors in bronchial epithelial cells: soluble ICAM-1 release inhibits human rhinovirus infection,” (2008) J Inflamm (Lond). 5: p. 8; Jartti et al., “Systemic T-helper and T-regulatory cell type cytokine responses in rhinovirus vs. respiratory syncytial virus induced early wheezing: an observational study,” (2009) Respir Res. 10: p. 85; Hershey et al., “The association of atopy with a gain-of-function mutation in the alpha subunit of the interleukin-4 receptor,” (1997) N Engl J Med. 337: pp. 1720-1725; Abdel-Hafez et al., “Idiopathic nephrotic syndrome and atopy: is there a common link?,” (2009) Am J Kidney Dis. 54: pp. 945-953.

Common colds, frequently caused by Rhinoviruses, trigger nearly 70% of episodes of relapse of the glomerular diseases MCD and FSGS. See Passioti et al., “The common cold: potential for future prevention or cure,” (2014) Curr Allergy Asthma Rep. 14: p. 413; Takahashi et al., “Triggers of relapse in steroid-dependent and frequently relapsing nephrotic syndrome,” (2007) Pediatr Nephrol. 22: pp. 232-236.

Whereas this relapse pathway is unknown, the inventors have long considered the cytokine storm to play a leading role. Since the COVID-19 cytokine storm is broader than its common cold counterpart, subtractive analysis could identify key players in specific aspects of each disease. Human and experimental MCD and most forms of FSGS are associated with low podocyte expression of transcriptional factor ZHX2. See Macé et al., “ZHX2 and its interacting proteins regulate upstream pathways in podocyte diseases,” (2020) Kidney Int. 97: pp. 753-764. By contrast, experimental evidence suggests that the collapsing variant of FSGS has high underlying podocyte ZHX2 expression. Macé et al. 2020. In contrast to many other cells, podocytes express the majority of all ZHX proteins in a cell membrane (non-nuclear) distribution. In the right combinations and the setting of an altered ZHX2 expression state, systemic cytokine release could potentially induce migration of ZHX proteins from normal (Aminopeptidase A/APA, Ephrin B1) or putative alternative cell membrane anchors into the podocyte nucleus.

However, despite efforts to understand the resultant cytokine storm seen following COVID-19 infection, there is still a need to understand the underlying mechanisms of this phenomenon and the resultant clinical manifestations. Such an understanding will facilitate the design of therapeutic approaches to reduce cytokine storm related organ damage.

The present invention addresses these needs.

### SUMMARY OF THE INVENTION

The current invention provides mechanisms targeting various cytokines. The invention describes specific combinations of cytokines or soluble factors that must be depleted to eliminate or reduce the effects of the cytokine storm including mortality and end organ injury.

Viral illnesses, including respiratory tract viruses like SARS-COV-1 and SARS-COV-2, have pathologic effects on non-respiratory tract organs even in the absence of obvious direct viral infection. In addition, illness caused by other respiratory and non-respiratory viruses such as influenza, parainfluenza, Respiratory Syncytial Virus, adenoviruses, enteroviruses, other coronaviruses, cytomegalovirus (CMV), Epstein Barr Virus (EBV), Middle East Respiratory

Syndrome (MERS), and Ebola also are known to cause cytokine storms as well as cause mortality and multiorgan injury.

To study and compare the role of viral cytokine storms in extra-pulmonary manifestations of SARS-COV-2, novel COVID-19 and cytokine combination “cocktails” were developed from clinical data and injected in mice. Previous studies by the inventors demonstrated effectiveness in a Rhinovirus common cold infection model using Common Cold cytokine combination “cocktails.”

In initial studies, the inventors utilized Zhx2^{flox/flox} and NPHS2-promoter driven Cre mice. However, they subsequently used BALB/cJ mice, an established model of the Zhx2 hypomorph state, and BALB/c mice (Zhx2+/+). See Macé et al. 2020; Perincheri et al., “Hereditary persistence of alpha-fetoprotein and H19 expression in liver of BALB/cJ mice is due to a retrovirus insertion in the Zhx2 gene.” (2005) Proc Natl Acad Sci USA 102: pp. 396-4011; Perincheri et al., “Characterization of the ETnII-alpha endogenous retroviral element in the BALB/cJ Zhx2 (Afr1) allele,” (2008) Mamm Genome 19: pp. 26-31; Gargalovic et al., “Quantitative trait locus mapping and identification of Zhx2 as a novel regulator of plasma lipid metabolism,” (2010) Circ Cardiovasc Genet. 3: pp. 60-67; Creasy et al., “Zinc Fingers and Homeoboxes 2 (Zhx2) Regulates Sexually Dimorphic Cyp Gene Expression in the Adult Mouse Liver,” (2016) Gene Expr. 17: pp. 7-17; Jiang et al., “Zhx2 (zinc fingers and homeoboxes 2) regulates major urinary protein gene expression in the mouse liver,” (2017) J Biol Chem 292: pp. 6765-6774 (2017); Erbilgin et al., “Transcription Factor Zhx2 Deficiency Reduces Atherosclerosis and Promotes Macrophage Apoptosis in Mice,” (2018) Arterioscler Thromb Vasc Biol. 38: pp. 2016-2027.

At low doses, COVID-19 cocktails, but not individual cytokines, induced glomerular injury and albuminuria in mice to mimic COVID-19 related proteinuria. The cytokine cocktails activated STAT6 signaling in cultured podocytes, which was reduced in CRISPR B Zhx2 hypomorph podocytes. Depletion of select single cytokines improved glomerular injury and albuminuria. At high doses, COVID-19 cocktails, but not individual cytokines, induced common clinical manifestations of SARS-COV-2 disease, including acute heart injury, myocarditis, pericarditis, liver and kidney injury, and high mortality in mice. STAT5, STAT6 and NFκB pathways were activated in these organs. Dual depletion after model induction of select combinations of TNF-α with IL-2 or IL-13 or IL-4 in BALB/c. In summary, systemic manifestations of viral cytokine storms, disease mechanisms and therapeutic principles to reduce morbidity and mortality were identified.

In embodiments of the current invention are provided methods of inhibiting, treating, or preventing the effects of cytokine storms as the result of viral infections in patients comprising inhibiting, neutralizing or depleting one or more cytokines from the patient.

In any embodiment, the cytokine storm can be induced by a viral infection caused by any respiratory or non-respiratory virus. Thus, the viral infection can be caused by viral illnesses, including respiratory tract viruses like SARS-COV-1 and SARS-COV-2, have pathologic effects on non-respiratory tract organs even in the absence of obvious direct viral infection. In addition, the viral infection can be caused by other respiratory and non-respiratory viruses such as influenza, parainfluenza, Respiratory Syncytial Virus, adenoviruses, enteroviruses, other coronaviruses, cytomegalovirus (CMV), Epstein Barr Virus (EBV), Middle East Respiratory Syndrome (MERS), and Ebola which also are known to cause cytokine storms as well as cause mortality and multiorgan injury.

In another embodiment, cytokine storms are caused by non-viral infections, such as those caused by bacteria, fungi or protozoa.

In still another embodiment, cytokine storms are of non-infectious etiology, such as those related to cancers or their treatment, organ transplantation, or related to changes in the stable cytokine milieu of systemic disorders like diabetes mellitus.

Furthermore, because the inventors discovered that the major difference between common colds (that cause mild disease) and more severe cytokine storm profiles caused by viral infections induced by for example, SARS-COV-2, is the presence of concomitant acute activation of an IL-4, IL-13 related “allergy pathway,” the current invention also includes viral infections that include concomitant and significant activation of the allergy pathway.

In some embodiments, in invention provides methods of depleting two or more cytokines in order to reduce the mortality caused by severe cytokine storms.

In other embodiments of the invention are provided methods of treating the effects of acute heart injury, acute liver injury and acute kidney injury used by cytokine storms. In some embodiments, the cytokine storms are caused by viral infections.

In still other embodiments of the invention are provided methods of reducing mortality caused by cytokine storms. In some embodiments, the cytokine storms are caused by viral infections.

In embodiments of the current disclosure, methods are provided to prevent multi-organ injury induced by a cytokine storm comprising the inhibition, neutralization, or depletion more than one cytokine.

In still other embodiments of the current invention are provided methods for treating or preventing the effects of post-acute sequelae of a SARS-Cov-2 infection comprising the inhibition, neutralization or depletion of one or more cytokines.

In embodiments of the current disclosure, methods are provided for preventing the relapse of a viral infection. In particular embodiments, the methods involve providing treatments that inhibit, neutralize or deplete one or more cytokines.

In yet other embodiments of the current invention are provided methods for treating or preventing the effects of SARS-COV-2 virus mRNA vaccines comprising the inhibition, neutralization or depletion of one or more cytokines.

In other embodiments are provided animal models for cytokine storms induced by viral infections and other disease states to test methods of treating or preventing the effects of said cytokine storm.

Thus, in any of the methods provided in the current invention, one or more cytokines can be inhibited, neutralized or depleted by the administration of and agent to the patient where the agent comprises an adeno-associated virus (AAV) or lentovirus-containing an a short-hairpin RNA (shRNA) against one or more cytokines.

In some embodiments, the shRNA is commercially available and can be attached to or part of any vector known in the art including plasmids, viral vectors, bacteriophages, cosmids, and artificial chromosomes.

In other embodiments, the agent comprises a monoclonal or polyclonal antibody directed against the one or more cytokines. In yet other embodiments, the agent comprises a monoclonal or polyclonal antibody directed against one or more cytokines. In still other embodiments, the agent is an siRNA or antisense oligonucleotide that targets one or more cytokines.

In still other embodiments, the agent is an antagonist that binds to a cytokine-mediated receptor and prevents the binding of one or more cytokines.

In embodiments of the current disclosure, methods are provided for treating a viral infection. In particular embodiments, the methods involve providing treatments that inhibit, neutralize or deplete one or more cytokines.

In any of the disclosed embodiments, the one or more cytokines to be inhibited, neutralized or depleted comprise TNFα, IL-2, IL-4, IL-13, IFN-γ or IL-6.

It will be understood for the disclosure herein that depending upon the severity of the viral infection or other condition being treated, the inhibition, neutralization or depletion more than one cytokine may be more effective that depletion of a single cytokine.

### BRIEF DESCRIPTION OF THE DRAWINGS

FIG. 1a-g depicts the development and characterization of COVID-19 cytokine storm models. (FIG. 1a) Schematic representation of COVID-19 induced cytokine storm in the context of human disease. (FIG. 1b) Composition of dose X of the COVID cocktails A to D. (FIG. 1c) Albuminuria after injecting different doses of Cocktail D into BALB/cJ mice (n=4 mice per group). X/2 is the threshold nephritogenic dose in BALB/cJ mice. (FIG. 1d) Albuminuria after injecting dose X of individual COVID cocktail components in BALB/cJ mice (n=4 mice per group). (FIG. 1e) Albuminuria after injecting COVID cocktails A to D dose X/2 in BALB/c mice (n=6 mice per group). (FIG. 1f) Albuminuria after injecting COVID cocktails A to D dose X/2 in BALB/cJ mice (n=6 mice per group). (FIG. 1g) Albuminuria after injecting BALB/c mice with intact Cocktail C dose X/2 or Cocktail C dose X/2 lacking individual components that target podocytes (n=6 mice per group). P<0.05; **P<0.01; ***P<0.001. All significant values are two-tail

FIG. 2a-i is an assessment of systemic injury induced by high dose of Cocktail D (3×) in BALB/c mice, compared with lower doses or individual components at dose 3×. (FIG. 2a) Acute myocardial injury assessed by cardiac Troponin I levels (cTPI3) levels (n=8 mice per group). (FIG. 2b) Acute liver injury assessed by alanine aminotransferase (ALT) activity levels (n=8 mice per group). (FIG. 2c) Acute kidney injury assessed by serum creatinine measured using mass spectrometry (n=8 mice per group). (FIG. 2d) Histological characterization of acute cardiac injury (n=3 mice per group) using H&E-stained sections from Cocktail D dose 3× injected mice. Myocytolysis (red arrows), inflammation (black arrows), fibril disruption (blue arrows), hypereosinophilia (green arrows) and pericarditis (orange arrow) were noted. (FIG. 2e) Histological characterization of acute liver injury (n=3 mice per group) using H&E-stained sections from Cocktail D dose 3× injected mice. Hepatocellular injury (red arrows), inflammation (black arrows), prominent Kupfer cells (green arrows), regenerative changes (yellow arrows) and peri-central vein injury (blue arrow) were noted. (FIG. 2f) Histological assessment of acute kidney injury (n=3 mice per group) using PAS-stained sections (columns 1, 2, 4) and kidney electron microscopy (column 3) from Cocktail D dose 3× injected mice. First three columns show proximal tubules, last column shows distal tubules. In proximal tubules, vacuolation (red arrows), brush border disruption (green arrows) and tubular degeneration (black arrows) were noted. In distal tubules, evidence of desquamation (blue arrows) was present. Foam cells were also noted (white arrows). Light microscopy scale bars 20 μm; Electron microscopy scale bars 2.66 μm. (FIG. 2g) Tables showing morphometric analysis of histological changes in the heart in BALB/c mice. (FIG. 2h) Tables showing morphometric analysis of histological changes in the liver in BALB/c mice. (FIG. 2i) Tables showing morphometric analysis of histological changes in the kidney in BALB/c mice. *P<0.05; **P<0.01; ***P<0.001, all values based on two-tail analysis.

FIG. 3a-h shows therapeutic strategies for the effect of mild and moderate cytokine storms on glomerular and systemic disease. All depleting antibodies or control IgG were injected intravenously one hour after model induction. (FIG. 3a) Albuminuria after induction of the Cocktail C model in BALB/c mice (n=6 mice per group; dose X/2), followed by control IgG or depleting antibodies. Groups arranges from left to right in order of efficacy. (FIG. 3b) Urine albumin to creatinine ratio at baseline and on Day 1 of the Cocktail D dose 1.8× model (n=8 BALB/c mice per group) after depleting one or more components with antibodies. (FIG. 3c) Serum cardiac Troponin I (cTPI3) levels on Day 1 of the Cocktail D dose 1.8× model (n=8 BALB/c mice per group) after depleting one or more components with antibodies. Control and Cocktail D 1.8×+IgG injected BALB/cJ mice are shown for comparison. (FIG. 3d) Serum ALT activity on Day 1 of the Cocktail D dose 1.8× model (n=8 BALB/c mice per group) after depleting one or more components with antibodies. Control and Cocktail D 1.8×+IgG injected BALB/cJ mice are shown for comparison. (FIG. 3e) Serum creatinine on Day 1 of the Cocktail D dose 1.8× model (n=8 BALB/c mice per group) after depleting one or more components with antibodies. Control and Cocktail D 1.8×+IgG injected BALB/cJ mice are shown for comparison. (FIG. 3f) Tables showing morphometric analysis and comparison of histological changes in the heart between control IgG and antibody treated BALB/c mice. (FIG. 3g) Tables showing morphometric analysis and comparison of histological changes in the liver between control IgG and antibody treated BALB/c mice. (FIG. 3h) Tables showing morphometric analysis and comparison of histological changes in the kidney between control IgG and antibody treated BALB/c mice. Morphometric analysis n=3 mice per group. *P<0.05; **P<0.01; ***P<0.001, all values based on two-tail analysis.

FIG. 4a-g shows possible therapeutic strategies for the effect of severe cytokine storms on systemic disease in BALB/c mice. Number of mice injected per group are shown in panel a. All depleting antibodies or control IgG were injected intravenously one hour after model induction. Large (Red) asterisk indicates universal mortality. (FIG. 4a) Mortality table for BALB/c mice injected with Cocktail D 3× with control IgG or depleting antibodies. Since mortality was higher with metabolic cage use (5/6) than without (2/6) in the Control IgG group, timed urine collection for albuminuria was not conducted in these studies. (FIG. 4b) Serum cardiac Troponin I (cTPI3) levels on Day 1 among survivors of Cocktail D 3× dose injected mice, followed by control IgG or depleting antibodies. (FIG. 4c) Serum ALT activity levels on Day 1 among survivors of Cocktail D 3× dose injected mice, followed by control IgG or depleting antibodies. (FIG. 4d) Serum creatinine levels on Day 1 among survivors of Cocktail D 3× dose injected mice, followed by control IgG or depleting antibodies. (FIG. 4e) Morphometric comparison of cardiac histology between control and cytokine depletion groups. (FIG. 4f) Morphometric comparison of liver histology between control and cytokine depletion groups. (FIG. 4g) Morphometric comparison of kidney histology between control and cytokine depletion groups. Morphometric analysis n=3 mice per group. *P<0.05; **P<0.01; ***P<0.001, all values based on two-tail analysis.

FIG. 5a-e shows the activation of signaling pathways by COVID cocktails and disease mechanisms. Examples of qualitative studies for NFκB/p-p65 (liver, 30 minutes), pSTAT6 (kidney 60 minutes) and pSTAT5 (heart, 15 minutes) activation by Western blot of whole organ protein extracts of mice (n=3 per group) injected with Cocktail D 3× or control saline. (FIG. 5b) Western blots of quantitative studies to assess activation of pSTAT6 signaling in wild type and ZHX2 hypomorph (CRISPR B) cultured human podocytes incubated with human counterparts of Cocktail C (final concentration×/100,000; n=3 plates per condition). (FIG. 5c) Densitometry of Western blot of Cocktail C incubated wild type and CRISPR B podocytes from panel b. (FIG. 5d) Albuminuria in Il4ra^{-/-} and control BALB/cJ mice after injecting Cocktail C dose X/2 (left panel), and percentage increase in Day 1 albuminuria from baseline (right panel) (n=5 to 8 mice per group). (FIG. 5e) Schematic for potential binding of COVID cocktail components to specific receptors previously described in glomerular endothelial cell, mesangial cells and podocytes, and feedback loops (red) between these cells. *P<0.05; **P<0.01; ***P<0.001, all values based on two-tail analysis, except right panel in FIG. 5d is one-tail analysis.

FIG. 6a-c shows ancillary human data and additional effects of Cytokine Cocktails. (FIG. 6a) Plasma IL-4Rα levels assessed by ELISA in general COVID-19 patients, age, sex and race matched healthy controls, and COVID-19 patients with proteinuria. Number of patient samples assayed is shown below. (FIG. 6b) Electron microscopy images of BALB/cJ mouse glomeruli on Day 1 after injection of Cocktail D dose X/2. Areas of focal foot process effacement (black arrows), endothelial vacuolation (green circles), and endothelial hypertrophy (blue circles) were noted. (FIG. 6c) Serum creatinine, assayed by Mass Spectrometry, is not increased in COVID cytokine cocktails dose X/2 models (BALB/c and BALB/cJ mice; n=6 mice per group). Scale bars 0.5 μm. *P<0.05; ***P<0.001.

FIG. 7a-g: (FIG. 7a) Plasma creatine kinase, a marker of skeletal muscle injury, in BALB/cJ mice (n=4 mice per group) 24 hours after injection of Cocktail D at different doses. (FIG. 7b) Serum Cardiac Troponin I level data derived from FIG. 2a, plotted again for higher resolution of lesser increase in levels among some single cytokine injected groups. (FIG. 7c) Serum ALT level data derived from FIG. 2b, plotted again for higher resolution of lesser increase in levels among some single cytokine injected groups. (FIG. 7d) 18-hour albuminuria in BALB/c mice injected with single cytokine dose 3×, corresponding to FIG. 2a-c. Given their high mortality after Cocktail D 3×, metabolic cage housing for timed urine collection is not feasible at this dose in BALB/c mice. (FIG. 7e) Electron microscopy of BALB/c mouse kidney glomeruli 24 hours after injection Cocktail D dose 3×. Extensive foot processes effacement (red arrows), endothelial hypertrophy (green arrows) and glomerular basement membrane (GBM) remodeling (blue arrows) were present. (FIG. 7f) Hematoxylin and Eosin-stained skeletal muscle from BALB/cJ mice 24 hours after injection of Cocktail D dose 3×. Focal inflammation (black arrows) was noted in some sections. (FIG. 7g) Albuminuria after induction of Cocktail C in BALB/c mice (n=6 mice per group; dose X/2), followed by receptor blockage using antibodies against IL-4Rα, TNFR1 and IL-10Rβ, or control IgG. Scale bars (e) 0.5 μm, (f) 20 μm. *P<0.05; **P<0.01; ***P<0.001.

FIG. 8a-e shows the histology of intermediate dose injury. Histological sections from studies of BALB/c mice (n=3 mice/group) euthanized 24 hours after Cocktail D dose 1.8× injection and additional antibodies or Control IgG injected one hour after model induction (see FIG. 3). The numbering code for each group is: “1”=Control IgG; “2”=Anti-TNFα Ab; “3”=Anti-IL-6 Abs; “4”=Anti-IL-10 Ab; “5”=Anti-TNFα+Anti-IFNγ+Anti-IL-4-Abs; “6”=Anti-IL4 Abs; “7”=Anti-TNFα+Anti-IL-4+Anti-IL-10 Abs; “8”=Anti-IFNγ Ab; “9”=Anti-TNFα+Anti-IL-4 Abs. (FIG. 8a) Two columns of H & E-stained sections of the heart and pericardium. Myocytolysis (red arrows), inflammation (black arrows), hypereosinophilia (green arrows), pericarditis (orange arrow) and pericardial microcalcification (blue arrow) were noted. (FIG. 8b) H & E-stained sections of the liver. Hepatocellular injury (red arrows), inflammation (black arrows), degenerative changes (green arrows), and regenerative changes (yellow arrows) were noted. (FIG. 8c) Toluidine blue stained epon sections of the kidney showing gross tubular morphology. Tubular vacuolation (red arows) and tubular degeneration (black arrows) were noted in proximal tubules. (FIG. 8d) Electron microscopy of kidney tubules. Tubular vacuolation (red arows) and tubular degeneration (black arrows) were noted in proximal tubules. (FIG. 8e) Electron microscopy of glomeruli. Areas of podocyte foot process effacement (black arrows) were noted. Scale bars (a) 20 μm (b) 20 μm (c) 20 μm (d) 0.5 μm (e) 0.5 μm.

FIG. 9a-d shows the histology for the severe injury model. Histological sections from studies of BALB/c mice (n=3 mice/group) euthanized 24 hours after Cocktail D dose 3× injection and additional antibodies or Control IgG injected one hour after model induction (see FIG. 4). The numbering code for each group is: “1”=Control IgG; “2”=Anti-IL-2 Ab; “3”=Anti-TNFα+Anti-IL-2 Abs; “4”=Anti-TNFα+Anti-IL-13 Abs; “5”=Anti-TNFα+Anti-IL-4-Abs; “6”=Anti-TNFα Ab; “7”=Anti-IL-13; “8”=Anti-IL-4 Ab; “9”=Anti-TNFα+Anti-IFNγ Abs; “10”=Anti-TNFα+Anti-IL-6 Abs; “11”=Anti-IFNγ Ab; “12”=Anti-TNFα+Anti-ACE2 Abs; “13”=Anti-TNFα+Anti-IL-10 Abs; “14”=Anti-IL-6 Ab. (FIG. 9a) Two columns of H & E stained sections of the heart and pericardium. Myocytolysis (red arrows), inflammation (black arrows), hypereosinophilia (green arrows) and pericarditis (orange arrow) were noted. (FIG. 9b) Two columns of H & E stained sections of the liver. Hepatocellular injury (red arrows), inflammation (black arrows), degenerative changes (green arrows), and regenerative changes (yellow arrows) were noted. (FIG. 9c) Two columns of Toluidine blue stained sections of the kidney showing gross tubular morphology. Tubular vacuolation (red arrows) and tubular degeneration (black arrows) were noted in proximal tubules. (FIG. 9d) Two columns of electron microscopy of the kidney showing images of glomeruli. Areas of podocyte foot process effacement (black arrows) were noted. Scale bars (a) 20 μm (b) 20 μm (c) 20 μm (d) 0.5 μm.

FIG. 10a-c: (FIG. 10a) Confocal expression of cytokine receptors in BALB/c mouse glomeruli. White arrows indicate receptor expression in podocytes (P), endothelial (E) and mesangial (M) cells. Since TNFR1 is expressed in podocytes and endothelial cells, only partial co-localization with nephrin (blue), a podocyte protein, is noted. Green color is nuclear stain. (FIG. 10b) Confocal expression (red) of ACE-2 and cytokine receptors in BALB/c mouse kidney tubules. Most images show proximal tubules, except IL-10Rβ image is collecting duct. (FIG. 10c) Western blot characterization of antibodies used for depletion studies using recombinant proteins that make up the cytokine cocktails. Scale bars (a) 20 μm (b) 20 μm.

FIG. 11a is a schematic representation of data assembled from the Human Protein Atlas Project (https://www.proteinatlas.org/) showing the approximate distribution and semi-quantitative expression of cytokine receptors and ACE2 kidney tubular segments (FIG. 11b) heart muscle and (FIG. 11c) liver.

### DETAILED DESCRIPTION

The current invention provides mechanisms targeting various cytokines. The invention describes specific combinations of cytokines or soluble factors that must be depleted to eliminate or reduce the effects of the cytokine storm including mortality and end organ injury.

### Summary of Methods Provided

The current invention provides methods of inhibiting, treating, or preventing the effects of cytokine storms as the result of viral infections in patients comprising inhibiting, neutralizing or depleting one or more cytokines from the patient.

The inventors contemplate that in any embodiment, the cytokine storm can be induced by a viral infection caused by any respiratory or non-respiratory virus. Thus, the viral infection can be caused by viral illnesses, including respiratory tract viruses like SARS-COV-1 and SARS-CoV-2, have pathologic effects on non-respiratory tract organs even in the absence of obvious direct viral infection. In addition, the viral infection can be caused by other respiratory and non-respiratory viruses such as influenza, parainfluenza, Respiratory Syncytial Virus, adenoviruses, enteroviruses, other coronaviruses, cytomegalovirus (CMV), Epstein Barr Virus (EBV), Middle East Respiratory Syndrome (MERS), and Ebola which also are known to cause cytokine storms as well as cause mortality and multiorgan injury. In addition, because the inventors discovered that the major difference between common colds (that cause mild disease) and more severe cytokine storm profiles caused by viral infections induced by for example, SARS-COV-2, is the presence of concomitant acute activation of an IL-4, IL-13 related “allergy pathway,” the current invention also includes viral infections that include concomitant and significant activation of the allergy pathway.

Embodiments of the invention provide:
methods of depleting two or more cytokines in order to reduce the mortality caused by severe cytokine storms;
methods of treating the effects of acute heart injury, acute liver injury and acute kidney injury used by cytokine storms; the inventors contemplate that the cytokine storms can be caused by viral infections in some embodiments;
methods of reducing mortality caused by cytokine storms; the inventors contemplate that the cytokine storms can be caused by viral infections in some embodiments;
methods to prevent multi-organ injury induced by a cytokine storm comprising the inhibition, neutralization, or depletion more than one cytokine.
methods for treating or preventing the effects of post-acute sequelae of a SARS-Cov-2 infection comprising the inhibition, neutralization or depletion of one or more cytokines.
methods for preventing the relapse of a viral infection where the methods involve providing treatments that inhibit, neutralize or deplete one or more cytokines;
methods for treating or preventing the effects of SARS-COV-2 virus mRNA vaccines comprising the inhibition, neutralization or depletion of one or more cytokines;
animal models for cytokine storms induced by viral infections and other disease states to test methods of treating or preventing the effects of said cytokine storm; and,
methods are for treating a viral infection.

The inventors also contemplate that the methods of the current invention can be used in any disease state in which a cytokine storm occurs including those diseases of non-viral origin such as bacterial, fungal or parasitic infections, cancer, organ transplantation, or results from the change in the systemic cytokine milieu of a multisystem disease like diabetes mellitus or metabolic syndrome.

The inventors contemplate that in any of the methods disclosed, one or more cytokines can be inhibited, neutralized or depleted one or more of several methods. One method contemplated is by the administration of an agent to the patient where the agent comprises an adeno-associated virus (AAV) or lentovirus-containing an a short-hairpin RNA (shRNA) against one or more cytokines. The shRNA can be made or is commercially available and can be attached to or part of any vector known in the art including plasmids, viral vectors, bacteriophages, cosmids, and artificial chromosomes.

Another method contemplated of depleting one or more cytokines is by the administration of a monoclonal or polyclonal antibody directed against the one or more cytokines. In yet other embodiments, the agent comprises a monoclonal or polyclonal antibody directed against one or more cytokines.

The agent can also be an siRNA or antisense oligonucleotide that targets one or more cytokines.

In still other embodiments, the agent is an antagonist that binds to a cytokine-mediated receptor and prevents the binding of one or more cytokines.

In any of the disclosed embodiments, the one or more cytokines to be inhibited, neutralized or depleted comprise TNFα, IL-2, IL-4, IL-13, IFN-γ or IL-6. It will be understood for the disclosure herein that depending upon the severity of the viral infection or other condition being treated, the inhibition, neutralization or depletion more than one cytokine may be more effective that depletion of a single cytokine.

Throughout this disclosure, various quantities, such as amounts, sizes, dimensions, proportions and the like, are presented in a range format. It should be understood that the description of a quantity in range format is merely for convenience and brevity and should not be construed as an inflexible limitation on the scope of any embodiment. Accordingly, the description of a range should be considered to have specifically disclosed all the possible subranges as well as all individual numerical values within that range unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 4.62, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “includes”, “comprises”, “including” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C).

Unless specifically stated or obvious from context, as used herein, the term “about” in reference to a number or range of numbers is understood to mean the stated number and numbers+/−10% thereof, or 10% below the lower listed limit and 10% above the higher listed limit for the values listed for a range.

In any of the embodiments disclosed herein, the terms “treating” or “to treat” includes restraining, slowing, stopping, or reversing the progression or severity of an existing symptom or disorder.

In any of the embodiments disclosed herein, the term “patient” refers to a human.

### Cytokine Inhibitors

The current invention contemplates that various cytokines can be neutralized or inhibited by several different non-limiting methods. For example, as described herein, target cytokines can be neutralized or inhibited by administration of a therapeutically effective amount of an agent where the agent comprises an adeno-associated virus (AAV) or lentovirus-containing an a short-hairpin RNA (shRNA) against one or more cytokines (sh-“cytokine”). In some embodiments, the sh-“cytokine” is commercially available and can be attached to or part of any vector known in the art including plasmids, viral vectors, bacteriophages, cosmids, and artificial chromosomes.

Alternatively, as described herein, the target cytokine or cytokines can be neutralized or inhibited by administration of a therapeutically effective amount of an agent where the agent comprises an antibody, bivalent antibody or a monoclonal antibody directed against the particular target cytokine or cytokines.

Further, as described herein, target cytokine or cytokines can be neutralized or inhibited by administration of a therapeutically effective amount of an agent where the agent comprises an siRNA or antisense oligonucleotide that targets target cytokine or cytokines.

Also, as contemplated herein, target cytokine or cytokines can be neutralized or inhibited by administration of a therapeutically effective amount of an agent where the agent comprises an antagonist that binds to a target cytokine-mediated receptor and prevents the binding of the target cytokine or cytokines.

The target cytokine inhibitor or inhibitors or a composition therein can be administered once per day, two or more times daily or once per week. The target cytokine inhibitor or inhibitors or composition containing the same can occur by any conventional means including orally intramuscularly, intraperitoneally or intravenously into the subject. If injected, they can be injected at a single site per dose or multiple sites per dose.

### Cytokine Antibodies and Related Inhibitors

More specifically a cytokine inhibitor is an antibody directed against any cytokine as disclosed herein. Examples of suitable antibodies directed against one or more target cytokines are disclosed herein and known to those of skill in the art. The cytokine antibody can also include an antibody fragment or a bivalent antibody or fragment thereof, inhibiting one or more target cytokines. As described herein, the cytokine inhibitor may be part of a pharmaceutical composition where the composition may include either an antibody or fragment thereof for one or more target cytokines.

The anti-cytokine antibodies described herein can be made or obtained by any means known in the art, including commercially. It is also contemplated that an antibody can be specifically reactive with a particular cytokine protein or polypeptide may also be used as an antagonist. An anti-cytokine antibody herein may be an antibody or fragment thereof that binds to a cytokine or a bivalent antibody that binds to two different cytokines.

As used herein, the term “antibody” refers to an immunoglobulin (Ig) whether natural or partly or wholly synthetically produced. The term also covers any polypeptide or protein having a binding domain which is, or is homologous to, an antigen-binding domain. The term further includes “antigen-binding fragments” and other interchangeable terms for similar binding fragments such as described below.

Native antibodies and native immunoglobulins are usually heterotetrameric glycoproteins of about 150,000 Daltons, composed of two identical light (L) chains and two identical heavy (H) chains. Each light chain is typically linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (“V_{H}” or “VH”) followed by a number of constant domains (“C_{H}” or “CH”). Each light chain has a variable domain at one end (“V_{L}” or “VL”) and a constant domain (“CL” or “CL”) at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

The cytokine inhibitors as described herein can be a “synthetic polypeptide” derived from a “synthetic polynucleotide” derived from a “synthetic gene,” meaning that the corresponding polynucleotide sequence or portion thereof, or amino acid sequence or portion thereof, is derived, from a sequence that has been designed, or synthesized de novo, or modified, compared to an equivalent naturally occurring sequence. Synthetic polynucleotides (antibodies or antigen binding fragments) or synthetic genes can be prepared by methods known in the art, including but not limited to, the chemical synthesis of nucleic acid or amino acid sequences. Synthetic genes are typically different from naturally occurring genes, either at the amino acid, or polynucleotide level, (or both) and are typically located within the context of synthetic expression control sequences. Synthetic gene polynucleotide sequences, may not necessarily encode proteins with different amino acids, compared to the natural gene; for example, they can also encompass synthetic polynucleotide sequences that incorporate different codons but which encode the same amino acid (i.e., the nucleotide changes represent silent mutations at the amino acid level).

With respect to anti-cytokine antibodies, the term “antigen” refers to the any of the cytokine proteins disclosed herein, respectively or any fragment of the protein molecules thereof.

The terms “antigen-binding portion of an antibody,” “antigen-binding fragment,” “antigen-binding domain,” “antibody fragment” or a “functional fragment of an antibody” are used interchangeably herein to refer to one or more fragments of an antibody that retain the ability to specifically bind to one or more cytokines.

It is contemplated that the cytokine antibodies may also include “diabodies” which refers to small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. See for example, EP 404,097; WO 93/11161; and Hollinger et al., Proc. Natl. Acad. Sci. USA 90:6444 6448 (1993).

It is contemplated that the cytokine antibodies may also include “chimeric” forms of non-human (e.g., murine) antibodies include chimeric antibodies which contain minimal sequence derived from a non-human Ig. For the most part, chimeric antibodies are murine antibodies in which at least a portion of an immunoglobulin constant region (Fc), typically that of a human immunoglobulin are inserted in place of the murine Fc. See for example, Jones et al., Nature 321:522-525 (1986); Reichmann et al., Nature 332:323-329 (1988); and Presta, Curr. Op. Struct. Biol., 2:593-596 (1992).

It is contemplated that the cytokine antibodies may also include a “monoclonal antibody” which refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations, which can include different antibodies directed against different determinants (epitopes), each monoclonal antibody is directed against a single determinant on the antigen. The modifier “monoclonal” indicates the character of the antibody as being obtained from a substantially homogeneous population of antibodies and is not to be construed as requiring production of the antibody by any particular method. For example, monoclonal antibodies can be made by a hybridoma method, recombinant DNA methods, or isolated from phage antibody.

As used herein, “immunoreactive” refers to binding agents, antibodies or fragments thereof that are specific to a sequence of amino acid residues on a cytokine protein (“binding site” or “epitope”), yet if are cross-reactive to other peptides/proteins, are not toxic at the levels at which they are formulated for administration to human use. The term “binding” refers to a direct association between two molecules, due to, for example, covalent, electrostatic, hydrophobic, and ionic and/or hydrogen-bond interactions under physiological conditions and including interactions such as salt bridges and water bridges and any other conventional binding means. The term “preferentially binds” means that the binding agent binds to the binding site with greater affinity than it binds unrelated amino acid sequences.

As used herein, the term “affinity” refers to the equilibrium constant for the reversible binding of two agents and is expressed as Kd. Affinity of a binding protein to a ligand such as affinity of an antibody for an epitope can be, for example, from about 100 nanomolar (nM) to about 0.1 nM, from about 100 nM to about 1 picomolar (pM), or from about 100 nM to about 1 femtomolar (fM). As used herein, the term “avidity” refers to the resistance of a complex of two or more agents to dissociation after dilution. Apparent affinities can be determined by methods such as an enzyme linked immunosorbent assay (ELISA) or any other technique familiar to one of skill in the art. Avidities can be determined by methods such as a Scatchard analysis or any other technique familiar to one of skill in the art.

“Epitope” refers to that portion of an antigen or other macromolecule capable of forming a binding interaction with the variable region binding pocket of an antibody.

The term “specific” refers to a situation in which an antibody will not show any significant binding to molecules other than the antigen containing the epitope recognized by the antibody. The term is also applicable where, for example, an antigen binding domain is specific for a particular epitope which is carried by a number of antigens, in which case the antibody will be able to bind to the various antigens carrying the epitope. The terms “preferentially binds” or “specifically binds” mean that the antibodies bind to an epitope with greater affinity than it binds unrelated amino acid sequences, and, if cross-reactive to other polypeptides containing the epitope, are not toxic at the levels at which they are formulated for administration to human use.

The term “binding” refers to a direct association between two molecules, due to, for example, covalent, electrostatic, hydrophobic, and ionic and/or hydrogen-bond interactions under physiological conditions and includes interactions such as salt bridges and water bridges, as well as any other conventional means of binding.

As contemplated herein, a target cytokine inhibitor may be generated through gene expression technology. The term “RNA interference” or “RNAi” refers to the silencing or decreasing of gene expression by siRNAs. It is the process of sequence-specific, posttranscriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

The term “siRNAs” refers to short interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

It is also contemplated that any cytokine gene can be silenced or “turned” off” through the use of CRISPR technology as disclosed herein in the Examples.

### “Post-Acute Sequelae”

“Post-acute sequelae” of SARS-COV-2 or COVID-19, also known as “long COVID,” is used herein to describe any of the long-term symptoms or effects described as part of the invention that might be experienced weeks to months after primary infection with SARS-COV-2, the virus that causes COVID-19.

### General Methods

### COVID and Common Cold Cytokine Cocktails, and Related Animal Studies

All animal studies conducted were approved by the IACUC at Rush University or the University of Alabama at Birmingham. All animals received humane treatment per protocol. Methods for Dynabead assisted mouse glomerular isolation, rat glomerular isolation by sieving, histological section tissue preservation, timed 18-hour urine collection in metabolic cages in the absence of food, assessment of albuminuria and proteinuria, real time PCR, confocal imaging, electron microscopy and sample processing, histology for light microscopy, Western blot and co-immunoprecipitation are previously described and known. The following were assayed using commercially available kits using serum samples; mouse ALT (BioVision K752-100), mouse cardiac Troponin I Type 3 (Novus Biologicals NBP3-00456), mouse Creatine Kinase (Abcam ab 155901) and human IL-4Rα ELISA (Abcam ab46022). The following antibodies were purchased for Western blot: anti-pSTAT6 (Cell Signaling Technology, Inc. Danvers MA, USA; cat #56554, 1:500 dilution); anti-STAT6 (Cell Signaling Technology, Inc. Cat #5397, 1:500 dilution). Antibodies against ZHX1, ZHX2 and ZHX3 are previously described25,37,38. Maće et al. 2020; Liu et al., “ZHX proteins regulate podocyte gene expression during the development of nephrotic syndrome,” (2006) J. Biol. Chem. 281: pp. 39681-39692; Clement et al., “Early changes in gene expression that influence the course of primary glomerular disease,” (2007) Kidney Int. 72: pp. 337-347.

All cytokines, soluble receptors, and antibodies were injected intravenously in rodents, and are listed below:

| | | | | Injected |
|---|---|---|---|---|
| Cytokine/Receptor | Rat | Mouse | Human | Antibody |
| IL-2 | + | + | + | + |
| IL-4Rα | + | + | + | + |
| IL-4 | + | + | + | + |
| IL-13 | + | + | N.A. | + |
| IL-6 | + | + | + | + |
| IL-10 | + | + | + | + |
| Interferon-γ | + | + | + | + |
| TNF-α | + | + | + | + |
| ACE-2 | + | + | + | + |
| ICAM-1 | + | + | + | N.A. |
| TNFRI | | | | + |
| IL-10Rβ | | | | + |
| Rat gamma globulin | | | | + |
| Syrian hamster | | | | + |
| gamma globulin | | | | |

Antibodies used for depletion studies were characterized by Western blot using the corresponding recombinant protein. Each dose of cytokine cocktail was dissolved in a final volume of 100 μL of sterile 0.9% saline. BALB/cJ (Jackson Labs) and BALB/c (Envigo) mice were purchased at age 8 weeks, acclimatized for 2 weeks, and baseline 18-hour urine collection and tail blood sampling conducted. An extra baseline urine collection was conducted for BALB/cJ mice. Most in vivo studies were conducted between age 10 and 15 weeks. Enpep^{-/-}; Zhx2^{def/def} in mixed background were obtained by interbreeding the F2 cross between Enpep^{-/-25} and Zhx2 deficient BALB/cJ mice. The nephritogenic dose spectrum of cytokine cocktails was established for BALB/cJ, BALB/c, IL4r^{-/-} mice (Jackson Labs). During mouse cytokine studies using threshold nephritogenic doses (BALB/cJ, BALB/c, Il4r^{-/-} in BALB/cJ background, X/2;), 100 μL of 0.9% saline was given intraperitoneally immediately after the intravenous cytokine cocktail dose to maintain intravascular hydration. Two additional intraperitoneal injection of 100 μL of 0.9% saline were given at 6 and 23 hours in the intermediate and high cocktail models. During cytokine depletion studies, different groups of mice received 50 μg of control IgG or the respective antibody or antibody combination intravenously 1 hour after the administration of the mouse cytokine cocktail.

### Mass Spectrometry Assay for Plasma Creatinine

Serum creatinine was measured by LC/MS/MS using an Agilent 1290 Infinity II LC system in combination with a 2×50 mm, 2 μm Tosoh Bioscience TSK-GEL amide-80 LC column, interfaced to an Agilent 6495 Triple Quadrupole. The oven temperature was fixed at 40° C. The mobile phase consisted of 10 mM ammonium acetate in LCMS-grade water (35%) and LCMS-grade acetonitrile (ACN; 65%). Synthetic creatinine (ranging from 20 μg/ml to 0.16 ug/ml; Sigma) and isotope-labeled creatinine (D3-creatinine, 10 μg/ml; Sigma) were used as standard and internal standard, respectively. Then, 10 ul of sample or standard was combined with 5 μl internal standard and 235 ul 100% ACN, vortexed and centrifuged at 4° C. for 15 min at 15000 rpm. The supernatant was transferred to a new tube with 200 μl 10 mM ammonium acetate and 65% acetonitrile in LCMS-grade water, vortexed, centrifuged at 4° C. for 15 min at 15000 rpm and subsequently measured. All samples were measured in duplicate.

### Sources of Human Genomic DNA and Human Kidney Biopsies

Genomic DNA samples from 36 patients with nephrotic syndrome, 33 control subjects, and 16 patients with diabetic nephropathy were obtained from the following sources (a) Immortalized monocytes from plasma of nephrotic syndrome patients at the University of Alabama at Birmingham obtained via an IRB approved protocol X080813001 for collecting DNA, blood and urine samples. (b) IRB approved study at the Instituto Nacional De Cardiologia in Mexico City (CONACYT 34751M, CONACYT 11-05, and DPAGA-UNAM IN-201902) that included archived kidney biopsies from patients with glomerular diseases or pre-implantation kidney biopsies from healthy living related kidney donors. (c) Archived kidney biopsy, IRB exempt, from Hospital Nacional Alberto Sabogal Essalud, Lima, Peru. (d) Archived human DNA of previously published FSGS patients43,44 from the Duke Molecular Physiology Institute with known mutations in podocyte expressed related genes. (e) Coriell Cell Repositories, that archive DNA from the 1000 Genomes Project and the HAPMAP Project. For analytical comparisons between cases and controls, the 1000 genomes project phase 3 Ensambl v84 was included as a single additional control.

### Agilent Custom Capture and High Throughput Illumina Sequencing

A custom capture sequencing panel was created to isolate the genomic interval between HAS2 and ZHX2 on Chromosome 8. The target interval was uploaded to the SureDesign website for Agilent SureSelect capture probe design and synthesis (Agilent Technologies, Santa Clara CA). Genomic DNA library preparation and interval capture was done using the QXT SureSelect kit as per the manufacturer's instructions (Agilent Technologies). The resulting DNA libraries were quantitated by QPCR (Kapa Biosystems, Wilmington MA) and sequenced on the Illumina HiSeq 2500 or NextSeq 500 with paired end 100 bp sequencing following standard protocols. Approximately 15 million sequences were obtained per reaction. FASTQ file generation was done using bcl2fastq converter from Illumina (Illumina, Inc., San Diego CA). Paired Illumina sequences compared with hg38 database (GRCh38.p13 Primary Assembly) using CLC Genomics software (Version 12, Qiagen, Venlo, the Netherlands). Insertion and deletions of 3 bp size or larger and a minimum of 20 sequence reads were selected for analysis. Fisher test comparison of insertions and deletions in study and control subjects was exported in Excel format, followed by software assisted and manual exclusion of all insertions and deletions present in controls. Only insertions and deletions that were subsequently confirmed using IGV browser software (Broad Institute, Boston MA) were included. Establishment of homozygosity required presence of the InDel in over 85% of sequences, and subsequent confirmation by IGV. Minor discrepancies (1-2 base pair position differences) in the site of the insertion or deletion were occasionally noted between the two software and were resolved by Sanger sequencing while designing CRISPR Cas9 studies. All genomic numbering is based on hg38 and CLC genomics software.

### Genome Editing in Cultured Human Podocytes Using CRISPR/Cas9

The basic methodology for CRISPR Cas9 is previously published. See Cong et al., “Multiplex Genome Engineering using CRISPR/Cas Systems,” (2013) Science 339: pp. 819-823. A single cell derived clone of cells was generated from an established early passage immortalized human podocyte cell line51 and used for genome editing studies. The oligonucleotides and primers used are listed in Table 4.

TABLE 4

| | Application | |
|---|---|---|
| | Generation of Crispr-cas9 | |
| Name | mutants | Primer/Probe Sequence |
| | CRISPR B | |
| G0016 | SgRNA generation | 5′-CAC CGA CTG GTA AAC CAC TTA GGG C-3′ |
| G0017 | SgRNA generation | 5′-AAA CGC CCT AAG TGG TTT ACC AGT C-3′ |
| K1145 | SgRNA plasmid sequencing | 5′-GCA TAT ACG ATA CAA GGC TGT TAG AGA G-3′ |
| K1195 | Donor plasmid | 5′-CGG GCC GGA TCC CTA GAT GTA GCA TTA CCA GGG TGG-3′ |
| K1196 | Donor plasmid | 5′-GGC CGA AGC TTG CAG AGA AGA TCA CGA TAG ATT AGA AGA TG- |
| | | 3′ |
| K1207 | Sequencing of donor | 5′-GGT TTC CTT GTT ATA TCA CCA G-3′ |
| | plasmid | |
| K1215 | Quickchange Mutagenesis | 5′-GCT CTA GGA TGA CTG GTA AAC CAC TTA GGG CAG TCG TCC CCA |
| | | GAC CTG GTC TGT GGC CTG TTA G-3′ |
| K1216 | Quickchange Mutagenesis | 5′-CTA ACA GGC CAC AGA CCA GGT CTG GGG ACG ACT GCC CTA AGT |
| | | GGT TTA CCA GTC ATC CTA GAG C-3′ |
| K1219 | Plasmid linear amplifica- | 5′-GAT TAT CTT TCT AGG GTT AAC GAA CTT CAA GTA ATC AAG AGC |
| | tion | AGC-3′ |
| K1220 | Plasmid linear amplifica- | 5′-CGC AGA CTA TCT TTC TAG GGT TAA CTT TGT AGA ATG CTT CTC |
| | tion | G-3′ |
| K1217 | Puromycin cassette | 5′-CGA GAA GCA TTC TAC AAA GTT AAC CCT AGA AAG ATA GTC TGC |
| | | G-3′ |
| K1218 | Puromycin cassette | 5′-GCT GCT CTT GAT TAC TTG AAG TTC GTT AAC CCT AGA AAG ATA |
| | | ATC-3′ |
| K1189 | Genome editing | 5′-ACA CTG ACG ACA TGG TTC |
| | | TAC AGT CTC TGA AAC ATA GAA |
| | | GGC AC-3′ |
| K1188 | Genome editing | 5′-TAC GGT AGC AGA GAC TTG GTC TGA GAA TCT AAT ACC GCT GAT |
| | | CTG-3′ |
| | CRISPR A | |
| G0003 | SgRNA generation | 5′-CAC CGA CCC ATC CAT ACA CTT ACC C-3′ |
| G0004 | SgRNA generation | 5′-AAA CGG GTA AGT GTA TGG ATG GGT C-3′ |
| K1145 | SgRNA plasmid sequencing | 5′-GCA TAT ACG ATA CAA GGC TGT TAG AGA G-3′ |
| K1140 | Donor plasmid | 5′-GGC GGC ACT AGT CTA GCT GGC TTG ACT TTA CAA GAC GAT TCC |
| | | ATC C-3′ |
| K1141 | Donor plasmid | 5′-GGG CGG ATC CCT GCA CTC AGT ATT CTG CAA GTC CTG TAG C-3′ |
| K1151 | Sequencing of donor | 5′-CGA TCT CCT GAC CTC AAG-3′ |
| | plasmid | 5′-GTG CCT GGC CTG TTA TGA TCT TCT TAC TCA TIT GAT AGC ACC |
| | | AGT GTC CTG AGA AAA ATA ACA TAT ACT |
| K1149 | Quickchange Mutagenesis | CCA TTA CCC ATC CAT ACA CTT ACC CAG GCA CTC ATT CAC CAT ATT |
| | | AAC TAG ATA GAC ACA TGA TGT TGC |
| | | TGC TCC TGT TGA TGA TAA CAA TGT TGA GG-3′ |
| | | 5′-CCT CAA CAT TGT TAT CAT CAA CAG GAG CAG CAA CAT CAT GTG |
| | | TCT ATC TAG TTA ATA TGG TGA ATG AGT |
| K1150 | Quickchange Mutagenesis | GAC TGG GTA AGT GTA TGG ATG GGT AAT GGA GTA TAT GTT ATT |
| | | TTT CTC AGG ACA CTG GTG CTA TCA AAT |
| | | GAG TAA GAA GAT CAT AAC AGG CCA GGA C-3′ |
| K1163 | Plasmid linear amplifica- | 5′-CGT CAC AAT ATG ATT ATC TTT CTA GGG TTA ACT AGA TAG ACA |
| | tion | CAT GAT GTT GCT GCT CC-3′ |
| K1164 | Plasmid linear amplifica- | 5′-CGT CAA TIT TAC GCA GAC TAT CTT TCT AGG GTT AAT ATG GTG |
| | tion | AAT GAG TGA CTG GG-3′ |
| K1153 | Puromycin cassette | 5′-CCC AGT CAC TCA TTC ACC ATA TTA ACC CTA GAA AGA TAG TCT |
| | | GCG TAA AAT TGA CG-3′ |
| K1154 | Puromycin cassette | 5′-GGA GCA GCA ACA TCA TGT GTC TAT CTA GTT AAC CCT AGA AAG |
| | | ATA ATC ATA TTG TGA CG-3′ |
| K1138 | Genome editing | 5′-ACA CTG ACG ACA TGG TTC TAC AGT TAT GAT CTT CTT ACT CAT |
| | | TTG ATA GCA CCA GTG TCC-3′ |
| K1139 | Genome editing | 5′-TAC GGT AGC AGA GAC TTG GTC TGA AAG GAG CAG TGT TGA TCT |
| | | AGA GAG AGC C-3′ |
| | Real time PCR | |
| H826 | Human ZHX2 forward primer | CGGAACTGGCTGAATCAGACT |
| H827 | Human ZHX2 reverse primer | CAGCACAGCAGTTCTAACAGACTT |
| P246 | FAM-MGB Probe | TGCAGAGGCTGGCCA |

### CRISPR B

Generation of the sgRNA plasmid: In order to introduce a 10 bp insertion (CACACACACA), sgRNA recognizing a specific site 45 bp downstream of the insertion site (Chr8-122,533,694-122,533,695) was designed d using the Benchling website (https://benchling.com). Oligos G0016 and G0017 were phosphorylated and annealed using T4 Polynucleotide Kinase (NEB), digested with Bbsl and ligated into pX330-U6-Chimeric BB-CBh-hSpCas9 plasmid (a gift from Feng Zhang, Addgene plasmid #42230) using T7 DNA ligase (New England Biolabs). The ligation product was treated with PlasmidSafe exonuclease (Epicentre) to prevent unwanted recombination products and then transformed into One Shot TOP10 cells (Invitrogen). Ten colonies were picked up and plasmids were isolated using QIAprep Spin Miniprep Kit (QIAgen). Plasmid DNA was sequenced using primer K1145 (see Table 4).

Generation of the donor plasmid: The human genomic sequence from patient E58-13 containing the insertion under study was amplified using KAPA HiFi HotStart PCR Kit (Kapa Biosystems), the specific patient genomic DNA and primers K1195 and K1196, and cloned into pBlueScript II KS+ vector between the BamHI and HindIII restriction sites. Plasmid DNA was sequenced using K1207 to confirm the presence of the insertion. A single mutation in the PAM sequence was made to prevent cutting of this donor template plasmid using Quikchange mutagenesis kit (Agilent Technologies) and primers K1215 and K1216, and the change confirmed by sequencing. Next, this plasmid was amplified in linear fashion using primers K1219 and K1220, and the PCR product digested with DpnI to remove any residual circular template plasmid. The antibiotic selection cassette (Puromycin resistance and truncated thymidine kinase) flanked by ITR sequences was amplified by PCR from PB-MV1 Puro-TK plasmid (Transposagen) using primers K1217 and K1218, and ligated with the linearized plasmid (see above) at a TTAA region 78 bp upstream of the insertion using Gibson assembly Master Mix (NEB). NEB® 5-alpha Competent E. coli cells were transformed with 2 μl of the assembly reaction product. Plasmid DNA from 10 colonies were isolated and sequenced using primer K1217 to confirm correct assembly.

Genome editing using sgRNA and donor plasmids: For in vitro replication of InDels found in kidney disease patients, cultured human podocytes derived from a single cell were transfected by electroporation (Biorad Gene Pulser Xcell™ Electroporation System, 0.2 cm cuvette, square wave mode, 150 V and 10 millisecond pulse) with the CRISPR/Cas9 vector containing the specific sgRNA, and a donor plasmid containing the donor sequence and the antibiotic selection cassette. Following removal of non-transfected cells by incubation with 1 μg/ml Puromycin Dihydrochloride (Gibco) for 15 days, 10 μg of Excision-only piggyBac transposase expression vector (Transposagene) was transfected for scarless removal of the antibiotic selection cassette. Four days after transfection, cells were incubated with 2.5 μM ganciclovir (Sigma) to remove cells with residual truncated thymidine kinase activity. Single cells were picked, clones established, genomic DNA extracted using QIAamp DNA Mini Kit (QIAgen) and the target region PCR amplified using Platinum HiFi DNA polymerase (Invitrogen) and primers K1189 and K1188. PCR products were gel purified using QIAquick Gel Extraction Kit (QIAgen), cloned into pCR2.1 vector using TA Cloning™ kit (Invitrogen) and the insert sequenced using the M13 Forward sequencing primer. Sequences were aligned with native podocyte genomic sequence and the donor template sequence by BLAST.

### CRISPR A

Overall methods were identical to those for CRISPR B, with the exception of primers and oligonucleotides used, and the following site specific details: An 8 bp insertion (TGGATGGA) was introduced at Chr 8-122,304,094-122,304,095), and the sgRNA designed to recognize a specific site 73 bp upstream of the insertion site. While generating the donor plasmid, the patient specific genomic DNA (patient SF3) was cloned into the pBlueScript II KS+ vector between the SpeI and BamHI sites. During Gibson assembly, the antibiotic resistance cassette was ligated with the linearized plasmid at a TTAA region 51 bp upstream of the insertion.

### In Vitro STAT6 Signaling Studies

Wild-type (precursor of CRISPR modified podocytes) and CRISPR-B podocytes were grown in RPMI 1640 media containing heat-inactivated 10% fetal bovine serum, 1% Insulin-Transferrin-Selenium (ITS-G, Thermo Fisher Scientific-catalog number 41400045) and 1% Penicillin-Steptomycin (Thermo Fisher Scientific, catalog number 15140122) at 330 C. Cells were sub-cultured and 50,000 cells/dish were seeded on 10 cm culture dishes at 370 C for 3 days. Next, culture media were exchanged with RPMI 1640 containing heat-inactivated 0.2% FBS and 1% Penicillin-Steptomycin. After 24 hr, cells were treated with Cocktail C or Common Cold Cocktail (X/100,000) for 10, 20 and 30 min. Proteins were isolated with RIPA buffer (Thermo Fisher Scientific, catalog number: 89900) containing protease inhibitor (Thermo Fisher Scientific, catalog number: A32953) and phosphatase inhibitor (Thermo Fisher Scientific, catalog number: A32957) (10 ml of RIPA buffer contained 1 tablet each of protease and phosphatase inhibitor). Protein concentration was assessed using the Bradford protein assay.

Human Plasma from COVID-19 and Control Patients for IL-4Rα Assay

Human plasma 100 μL aliquots were obtained from the following sources (a) De-identified IRB approved hospitalized COVID patient samples from the Rush University COVID-19 Registry and Biorepository. (b) De-identified IRB approved hospitalized COVID patient samples from the Rush University COVID-19 Registry and Biorepository, selected for presence of proteinuria. (c) De-identified plasma samples that were age, sex and race matched to group a, purchased from Zenbio (Durham NC, USA).

### Statistical Analysis

Values in all graphs are mean+s. e. m. For difference in proteinuria, albuminuria or gene expression involving 2 groups, we used the unpaired Student's t test in Microsoft Excel 2013. Unless specifically indicated, all significance is two-tail.

Further reference is made to the following experimental examples.

### EXAMPLES

The following examples are provided for the purpose of illustrating various embodiments of the invention and are not meant to limit the present disclosure in any fashion. The present examples, along with the methods described herein are presently representative of preferred embodiments, are provided only as examples, and are not intended as limitations on the scope of the invention. Changes therein and other uses which are encompassed within the spirit of the disclosure as defined by the scope of the claims will occur to those skilled in the art.

### Example 1

### Developing Novel COVID-19 Cytokine Storm Cocktails

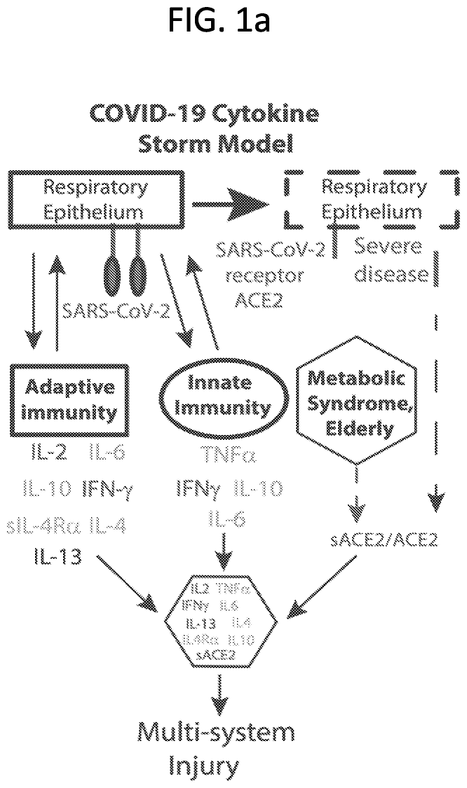

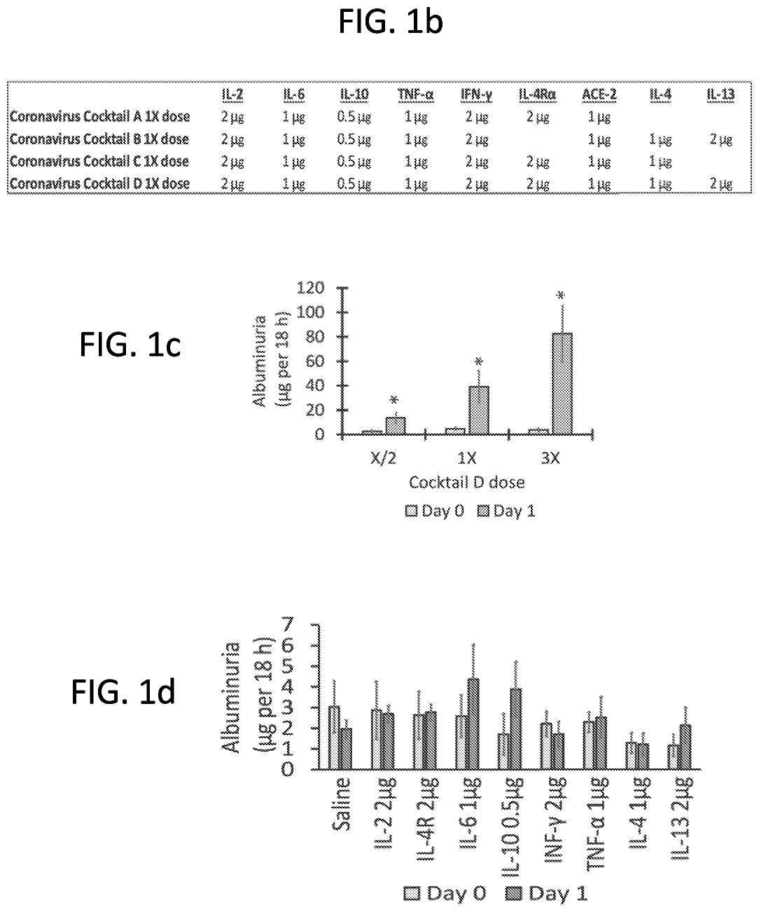

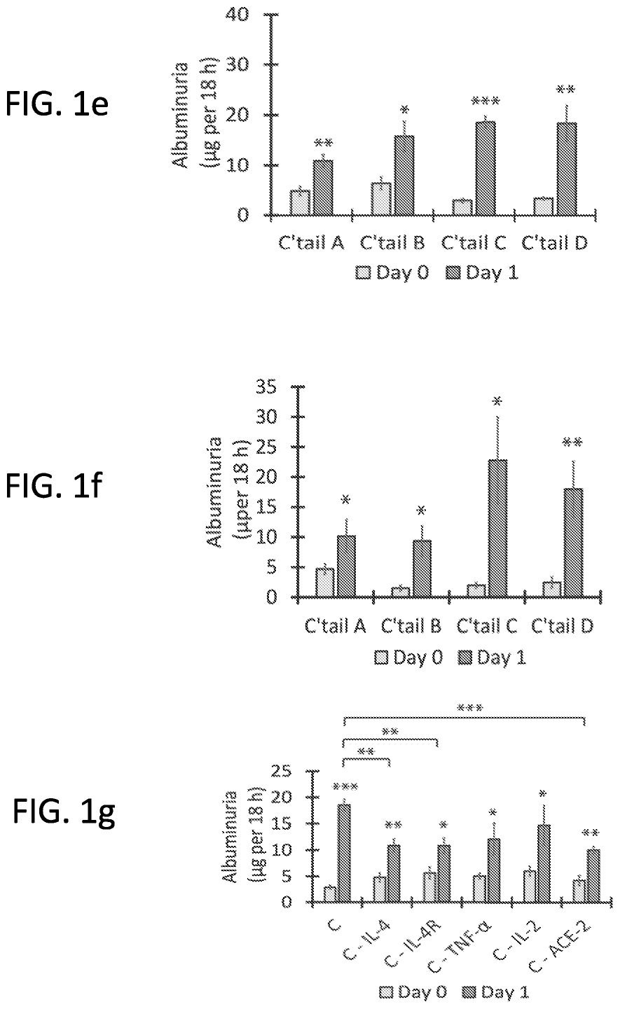

FIG. 1a-g depicts the development and characterization of COVID-19 cytokine storm models. (FIG. 1a) Schematic representation of COVID-19 induced cytokine storm in the context of human disease. (FIG. 1b) Composition of dose X of the COVID cocktails A to D. (FIG. 1c) Albuminuria after injecting different doses of Cocktail D into BALB/cJ mice (n=4 mice per group). X/2 is the threshold nephritogenic dose in BALB/cJ mice. (FIG. 1d) Albuminuria after injecting dose X of individual COVID cocktail components in BALB/cJ mice (n=4 mice per group). (FIG. 1e) Albuminuria after injecting COVID cocktails A to D dose X/2 in BALB/c mice (n=6 mice per group). (FIG. 1f) Albuminuria after injecting COVID cocktails A to D dose X/2 in BALB/cJ mice (n=6 mice per group). (FIG. 1g) Albuminuria after injecting BALB/c mice with intact Cocktail C dose X/2 or Cocktail C dose X/2 lacking individual components that target podocytes (n=6 mice per group). P<0.05; **P<0.01; ***P<0.001. All significant values are two-tail.

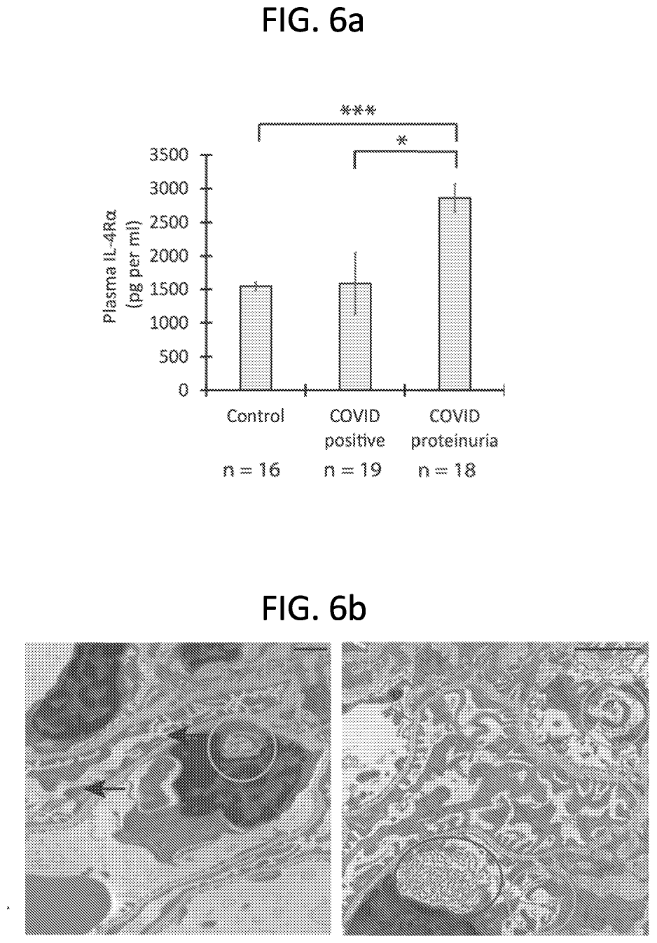

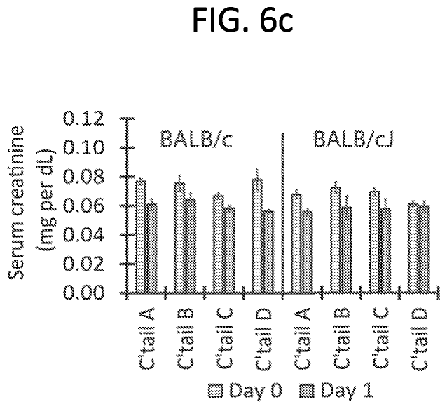

FIG. 6a-c shows ancillary human data and additional effects of Cytokine Cocktails. (FIG. 6a) Plasma IL-4Rα levels assessed by ELISA in general COVID-19 patients, age, sex and race matched healthy controls, and COVID-19 patients with proteinuria. Number of patient samples assayed is shown below. (FIG. 6b) Electron microscopy images of BALB/cJ mouse glomeruli on Day 1 after injection of Cocktail D dose X/2. Areas of focal foot process effacement (black arrows), endothelial vacuolation (green circles), and endothelial hypertrophy (blue circles) were noted. (FIG. 6c) Serum creatinine, assayed by Mass Spectrometry, is not increased in COVID cytokine cocktails dose X/2 models (BALB/c and BALB/cJ mice; n=6 mice per group). Scale bars 0.5 μm. *P<0.05; ***P<0.001.

COVID cocktails A to D were developed in a stepwise manner to model the hospitalized COVID-19 patients in intensive care (FIG. 1b, c). The first 5 cytokines (FIG. 1b) are common to all cocktails. Circulating IL-4Rα levels are also increased in COVID patients with proteinuria (FIG. 6a) ACE2, the COVID-19 receptor, was included in COVID-19 cocktails since plasma sACE2 levels are significantly higher in sick COVID-19 patients in Intensive Care, and in elderly and metabolic syndrome patients who are predisposed to severe COVID-19 disease. High plasma IL-13 and IL-4 levels in sick COVID-19 patients points towards acute activation of the allergy pathway in this disease. Removing sIL-4Rα from Cocktail A and adding IL-4 and IL-13 made cocktail B, whereas adding IL-4 to Cocktail A gave Cocktail C. Adding IL-13 to Cocktail C gave Cocktail D.

### Example 2

### Systemic Manifestations of Synergistic Multi-Cytokine Injury Induced by COVID-19 Cocktails

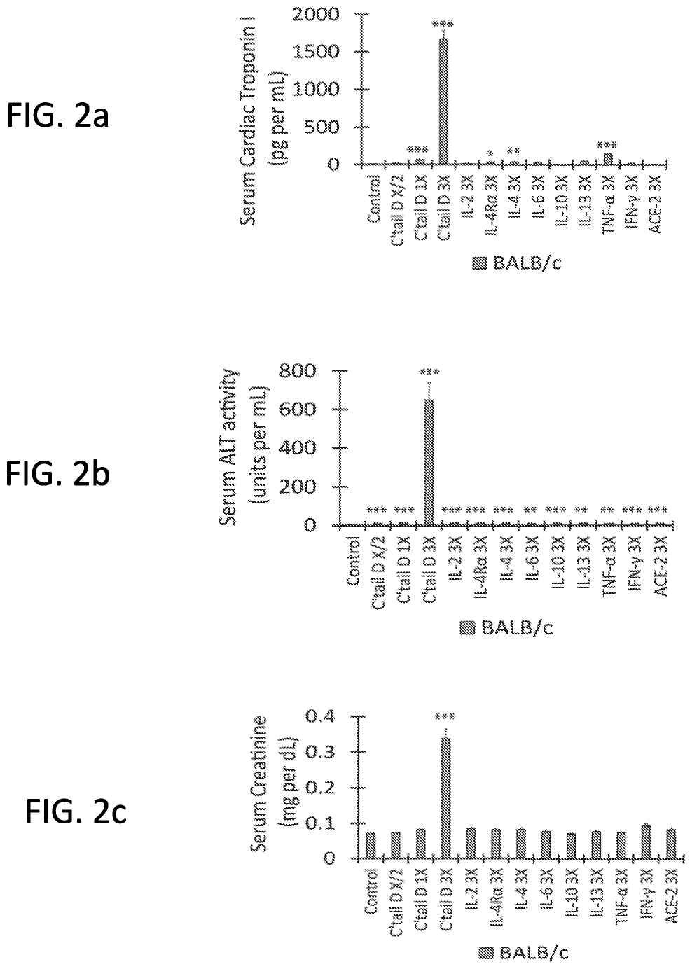

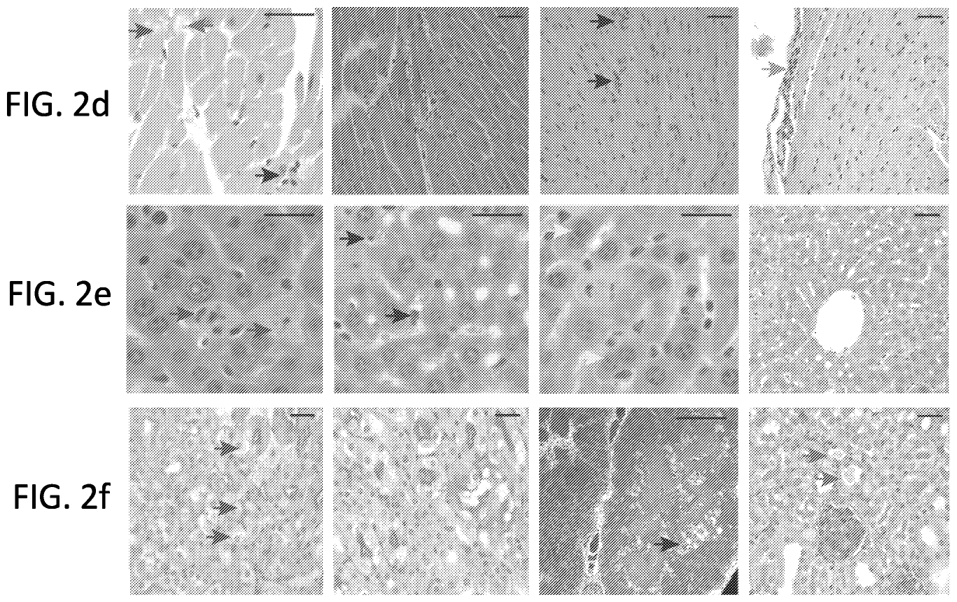

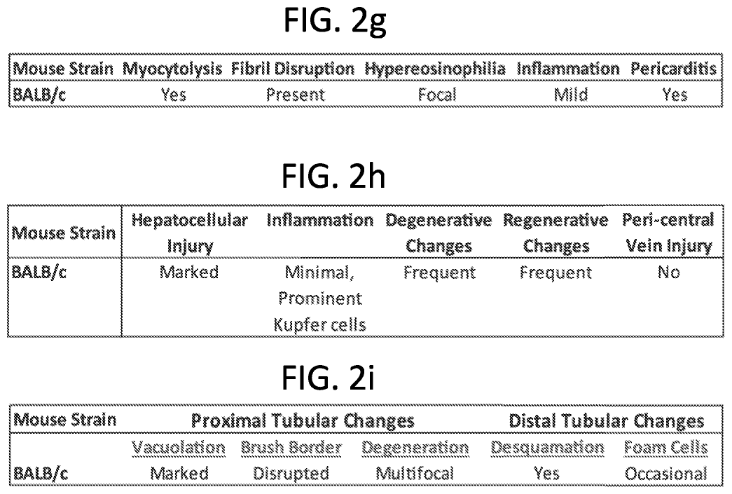

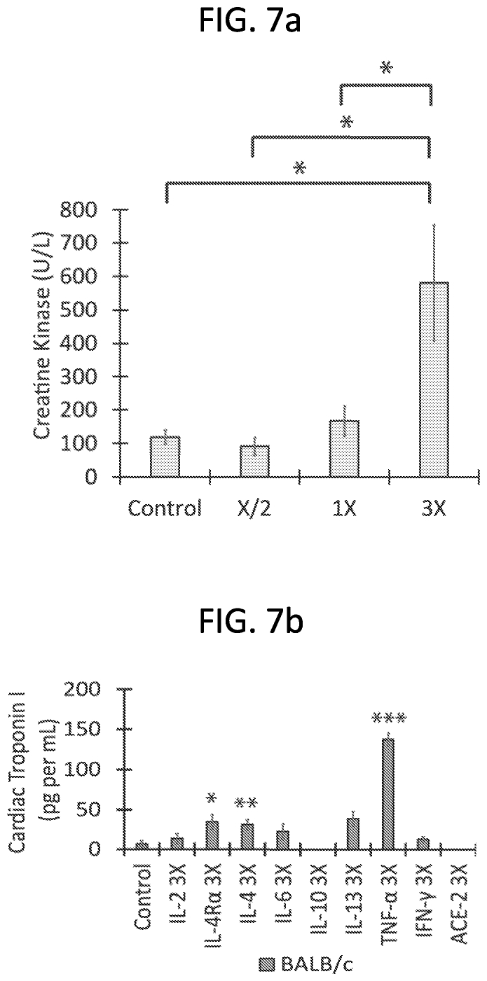

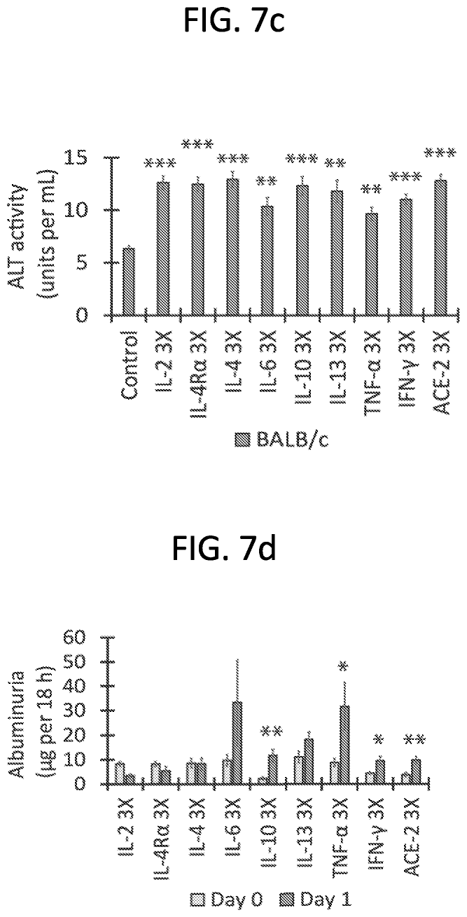

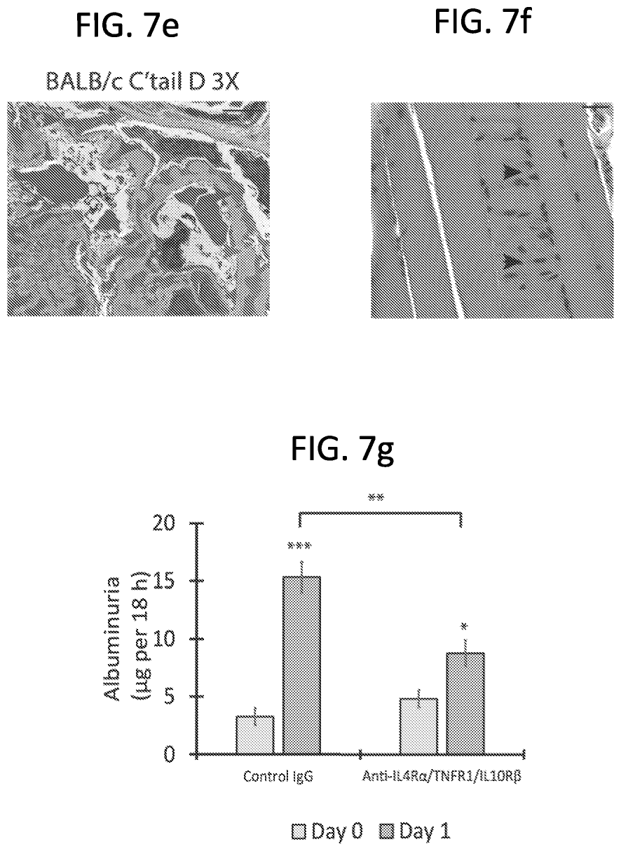

Injection of higher doses (3×) of Cocktail D induced albuminuria as well as causing elevation of serum cardiac Troponin I Type 3 (cTPI3; myocardial injury, FIG. 2a), serum alanine aminotransferase (ALT, acute liver injury, FIG. 2b), serum creatinine (Acute Kidney Injury, AKI; FIG. 2c), and plasma creatine kinase (CK, skeletal muscle injury; FIG. 7a).

FIG. 2a-i is an assessment of systemic injury induced by high dose of Cocktail D (3×) in BALB/c mice, compared with lower doses or individual components at dose 3×. (FIG. 2a) Acute myocardial injury assessed by cardiac Troponin I levels (cTPI3) levels (n=8 mice per group). (FIG. 2b) Acute liver injury assessed by alanine aminotransferase (ALT) activity levels (n=8 mice per group). (FIG. 2c) Acute kidney injury assessed by serum creatinine measured using mass spectrometry (n=8 mice per group). (FIG. 2d) Histological characterization of acute cardiac injury (n=3 mice per group) using H&E-stained sections from Cocktail D dose 3× injected mice. Myocytolysis (red arrows), inflammation (black arrows), fibril disruption (blue arrows), hypereosinophilia (green arrows) and pericarditis (orange arrow) were noted. (FIG. 2e) Histological characterization of acute liver injury (n=3 mice per group) using H&E-stained sections from Cocktail D dose 3× injected mice. Hepatocellular injury (red arrows), inflammation (black arrows), prominent Kupfer cells (green arrows), regenerative changes (yellow arrows) and peri-central vein injury (blue arrow) were noted. (FIG. 2f) Histological assessment of acute kidney injury (n=3 mice per group) using PAS-stained sections (columns 1, 2, 4) and kidney electron microscopy (column 3) from Cocktail D dose 3× injected mice. First three columns show proximal tubules, last column shows distal tubules. In proximal tubules, vacuolation (red arrows), brush border disruption (green arrows) and tubular degeneration (black arrows) were noted. In distal tubules, evidence of desquamation (blue arrows) was present. Foam cells were also noted (white arrows). Electron microscopy scale bars BALB/c, 2.66 μm; (FIG. 2g) Tables showing morphometric analysis of histological changes in the heart in BALB/c mice. (FIG. 2h) Tables showing morphometric analysis of histological changes in the liver in BALB/c mice. (FIG. 2i) Tables showing morphometric analysis of histological changes in the kidney in BALB/c mice. Light microscopy scale bars 20 μm. *P<0.05; **P<0.01; ***P<0.001, all values based on two-tail analysis.

FIG. 7a-g: (FIG. 7a) Plasma creatine kinase, a marker of skeletal muscle injury, in BALB/cJ mice (n=4 mice per group) 24 hours after injection of Cocktail D at different doses. (FIG. 7b) Serum Cardiac Troponin I level data derived from FIG. 2a, plotted again for higher resolution of lesser increase in levels among some single cytokine injected groups. (FIG. 7c) Serum ALT level data derived from FIG. 2b, plotted again for higher resolution of lesser increase in levels among some single cytokine injected groups. (FIG. 7d) 18-hour albuminuria in BALB/c mice injected with single cytokine dose 3×, corresponding to FIG. 2a-c. Given their high mortality after Cocktail D 3×, metabolic cage housing for timed urine collection is not feasible at this dose in BALB/c mice. (FIG. 7e) Electron microscopy of BALB/c mouse kidney glomeruli 24 hours after injection Cocktail D dose 3×. Extensive foot processes effacement (red arrows), endothelial hypertrophy (green arrows) and glomerular basement membrane (GBM) remodeling (blue arrows) were present. (FIG. 7f) Hematoxylin and Eosin-stained skeletal muscle from BALB/cJ mice 24 hours after injection of Cocktail D dose 3×. Focal inflammation (black arrows) was noted in some sections. (FIG. 7g) Albuminuria after induction of Cocktail C in BALB/c mice (n=6 mice per group; dose X/2), followed by receptor blockage using antibodies against IL-4Rα, TNFR1 and IL-10Rβ, or control IgG. Scale bars (e) 0.5 μm, (f) 20 μm. *P<0.05; **P<0.01; ***P<0.001.

cTPI3, ALT and albuminuria also increased at 3× dose for some individual cytokines, albeit at a significantly lower level than the cocktail (FIG. 2a, b; FIG. 7b-e). Timed urine collection in metabolic cages for albuminuria assessment was not conducted for Cocktail D 3× dose injected BALB/c mice in view of high mortality (see below). Cocktail D 3× dose induced severe cardiac, liver and acute kidney injury in BALB/c mice. Cardiac histology (FIG. 2d) revealed myocytolysis, focal fibrillar disruption and hypereosinophilia, inflammation (myocarditis) and pericarditis. Liver histology (FIG. 2e) showed substantial hepatocellular injury, prominent Kupfer cells, frequent degenerative and regenerative changes, and mild inflammation. Histological evaluation of the kidney tubulo-interstitial compartment (FIG. 2f) revealed evidence of proximal tubular injury in the form of frequent vacuolation, luminal widening, brush border disruption and desquamation of tubular epithelial cells. Desquamation of epithelial cells, foam cells and vacuolation were also noted in distal tubules. Morphometric changes in these organs 24 hours after injection of Cocktail D 3× in BALB/cJ mice are also described in FIG. 2g, h, i. No evidence of severe or extensive inflammation was seen.

### Example 3

### Therapeutic Cytokine Depletion to Disrupt Synergy in Mild and Intermediate Cytokine Storms

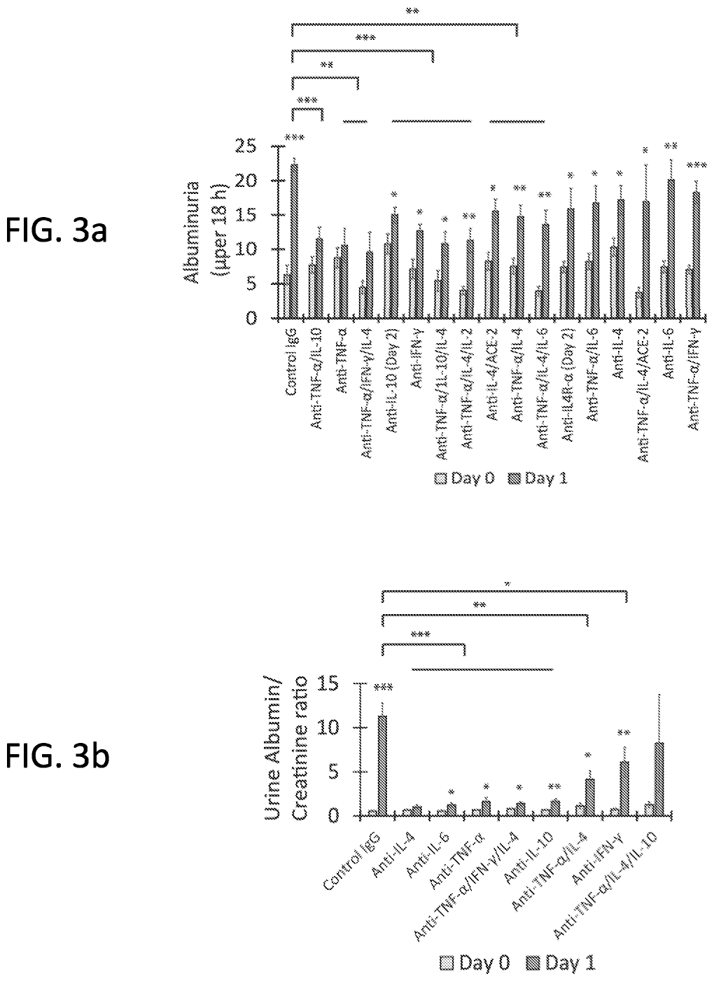

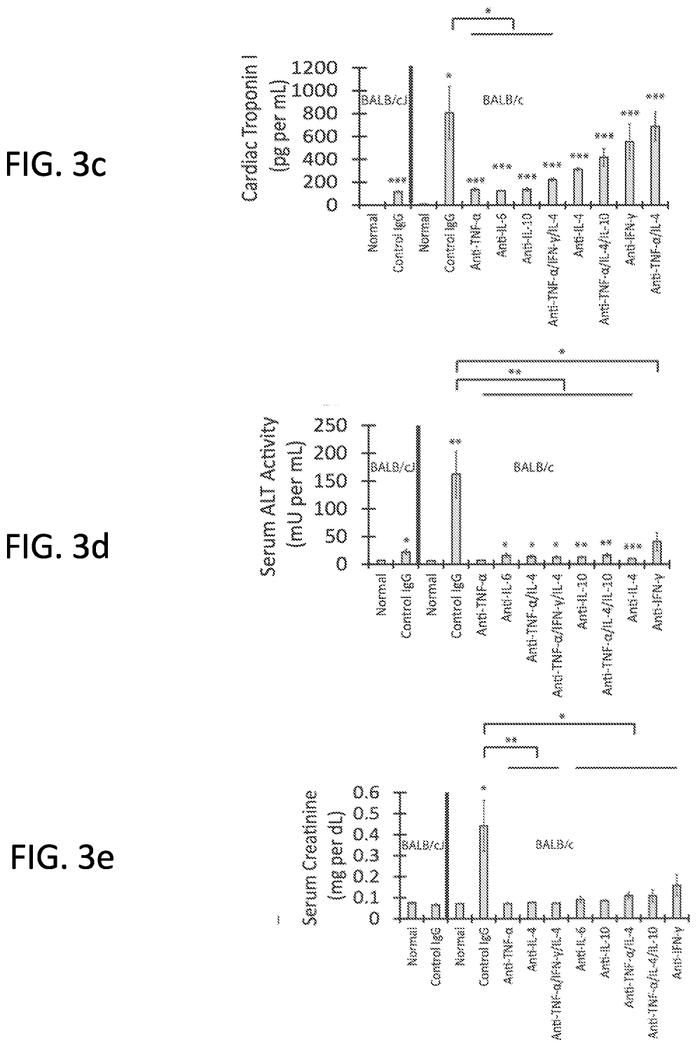

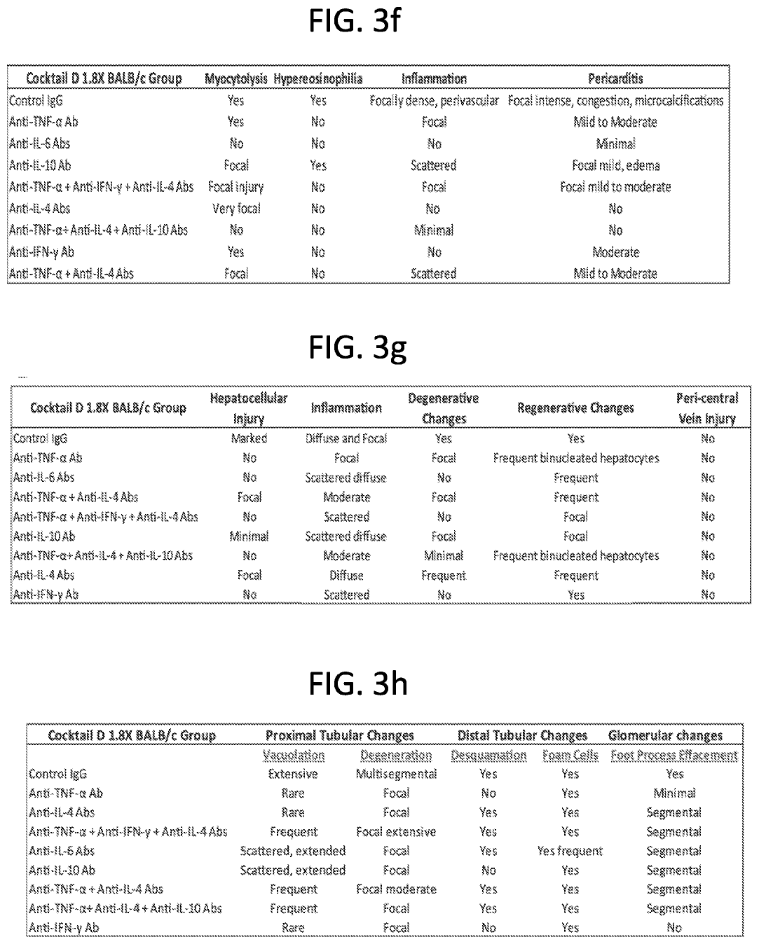

FIG. 3a-h shows therapeutic strategies for the effect of mild and moderate cytokine storms on glomerular and systemic disease. All depleting antibodies or control IgG were injected intravenously one hour after model induction. (FIG. 3a) Albuminuria after induction of the Cocktail C model in BALB/c mice (n=6 mice per group; dose X/2), followed by control IgG or depleting antibodies. Groups arranges from left to right in order of efficacy. (FIG. 3b) Urine albumin to creatinine ratio at baseline and on Day 1 of the Cocktail D dose 1.8× model (n=8 BALB/c mice per group) after depleting one or more components with antibodies. (FIG. 3c) Serum cardiac Troponin I (cTPI3) levels on Day 1 of the Cocktail D dose 1.8× model (n=8 BALB/c mice per group) after depleting one or more components with antibodies. Control and Cocktail D 1.8×+IgG injected BALB/cJ mice are shown for comparison. (FIG. 3d) Serum ALT activity on Day 1 of the Cocktail D dose 1.8× model (n=8 BALB/c mice per group) after depleting one or more components with antibodies. Control and Cocktail D 1.8×+IgG injected BALB/cJ mice are shown for comparison. (FIG. 3e) Serum creatinine on Day 1 of the Cocktail D dose 1.8× model (n=8 BALB/c mice per group) after depleting one or more components with antibodies. Control and Cocktail D 1.8×+IgG injected BALB/cJ mice are shown for comparison. (FIG. 3f) Tables showing morphometric analysis and comparison of histological changes in the heart between control IgG and antibody treated BALB/c mice. (FIG. 3g) Tables showing morphometric analysis and comparison of histological changes in the liver between control IgG and antibody treated BALB/c mice. (FIG. 3h) Tables showing morphometric analysis and comparison of histological changes in the kidney between control IgG and antibody treated BALB/c mice. Morphometric analysis n=3 mice per group. *P<0.05; **P<0.01; ***P<0.001, all values based on two-tail analysis.

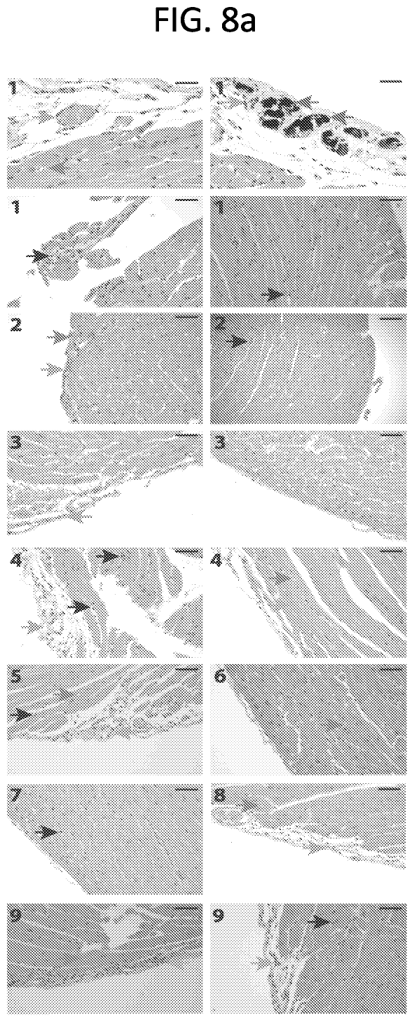

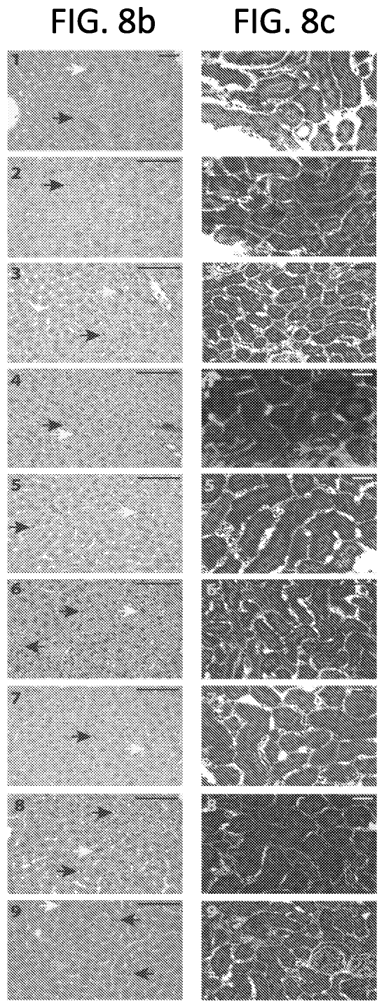

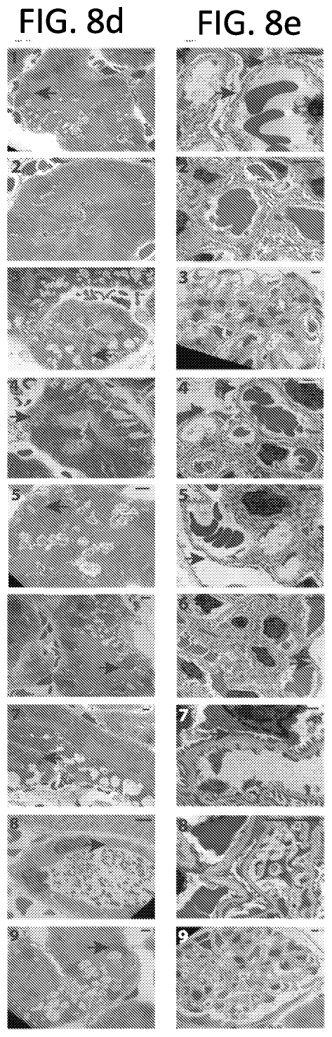

FIG. 8a-e shows the histology of intermediate injury. Histological sections from studies of BALB/c mice (n=3 mice/group) euthanized 24 hours after Cocktail D dose 1.8× injection and additional antibodies or Control IgG injected one hour after model induction (see FIG. 3). The numbering code for each group is: “1”=Control IgG; “2”=Anti-TNFα Ab; “3”=Anti-IL-6 Abs; “4”=Anti-IL-10 Ab; “5”=Anti-TNFα+Anti-IFNγ+Anti-IL-4-Abs; “6”=Anti-IL4 Abs; “7”=Anti-TNFα+Anti-IL-4+Anti-IL-10 Abs; “8”=Anti-IFNγ Ab; “9”=Anti-TNFα+Anti-IL-4 Abs. (FIG. 8a) Two columns of H & E-stained sections of the heart and pericardium. Myocytolysis (red arrows), inflammation (black arrows), hypereosinophilia (green arrows), pericarditis (orange arrow) and pericardial microcalcification (blue arrow) were noted. (FIG. 8b) H & E-stained sections of the liver. Hepatocellular injury (red arrows), inflammation (black arrows), degenerative changes (green arrows), and regenerative changes (yellow arrows) were noted. (FIG. 8c) Toluidine blue stained epon sections of the kidney showing gross tubular morphology. Tubular vacuolation (red arrows) and tubular degeneration (black arrows) were noted in proximal tubules. (FIG. 8d) Electron microscopy of kidney tubules. Tubular vacuolation (red arows) and tubular degeneration (black arrows) were noted in proximal tubules. (FIG. 8e) Electron microscopy of glomeruli. Areas of podocyte foot process effacement (black arrows) were noted. Scale bars (a) 20 μm (b) 20 μm (c) 20 μm (d) 0.5 μm (e) 0.5 μm.

Injecting a low (X/2) dose of Cocktail C (FIG. 3a) followed by single or combination cytokine depletion, in BALB/c mice showed significant reduction in albuminuria by anti-TNF-α, anti-IL-10, anti-IFN-γ, and select anti-TNFα antibody-based combinations. In many cases, depleting more cytokines was not always better, especially in mild cytokine storm models, suggesting that over-manipulation of the cytokine milieu can be counter-productive. In the intermediate dose (1.8×) model with Cocktail D in BALB/c mice, anti-IL-4, anti-IL-6, TNF-α, and some anti-TNFα antibody-based combinations were effective in reducing albuminuria (FIG. 3b), cTPI3 (except anti-IL-4, FIG. 3c), serum ALT levels (FIG. 3d) and normalizing serum creatinine (FIG. 3e). Morphometric analysis of these studies showed significant improvement in histological changes in the most effective regimens discussed above. (FIG. 3f-h; FIG. 8a-e).

### Example 4

### Therapeutic Cytokine Depletion to Disrupt Synergy, Prevent Mortality and Reduce Multi-Organ Toxicity in Severe Cytokine Storms

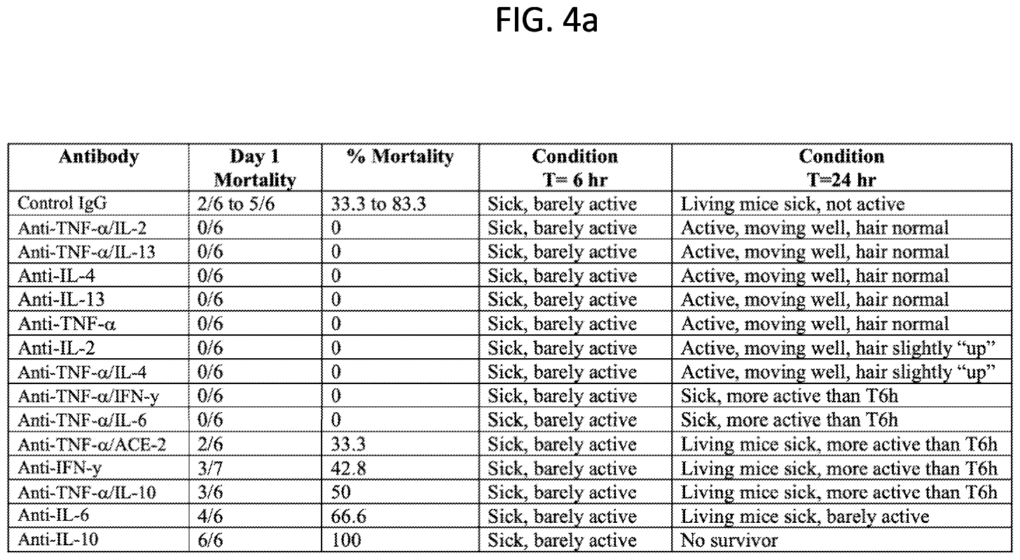

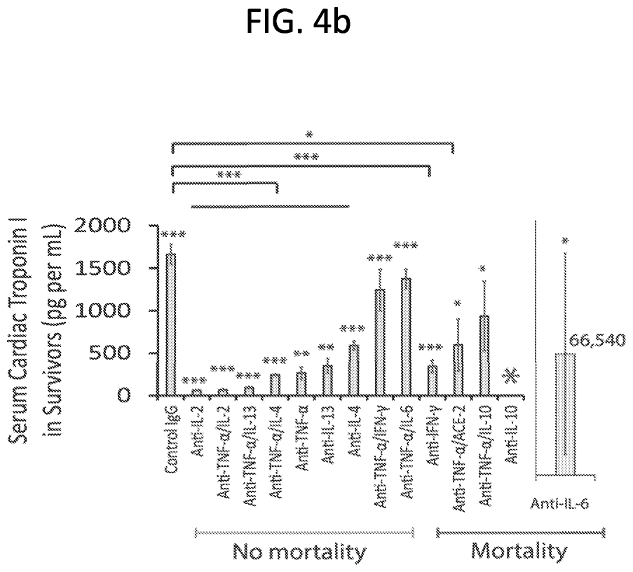

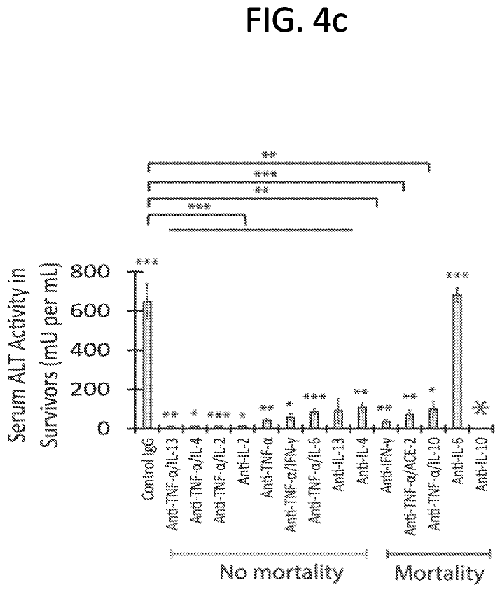

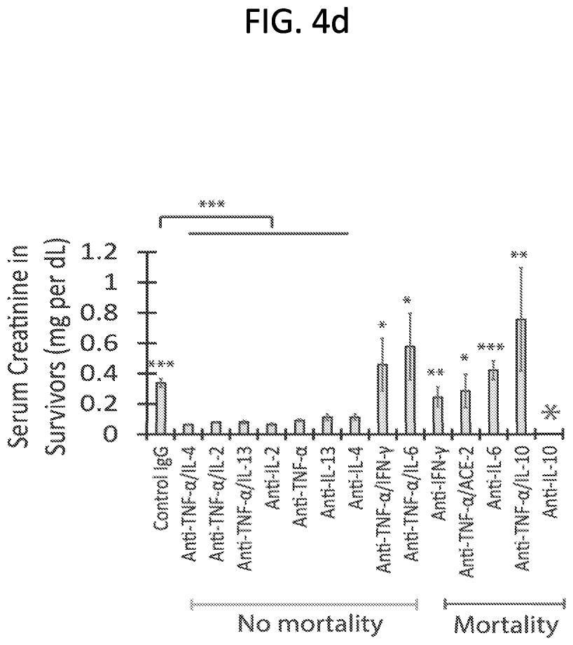

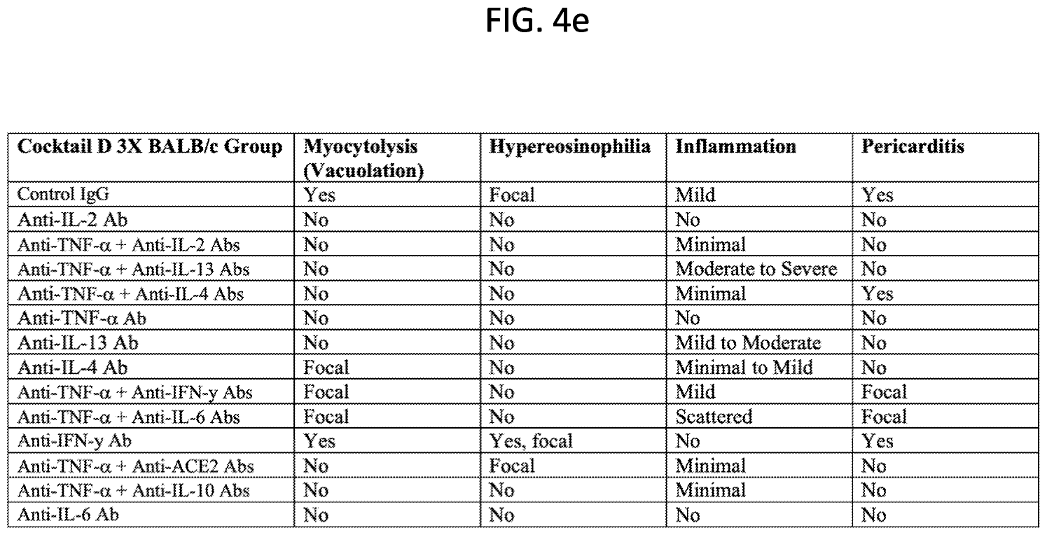

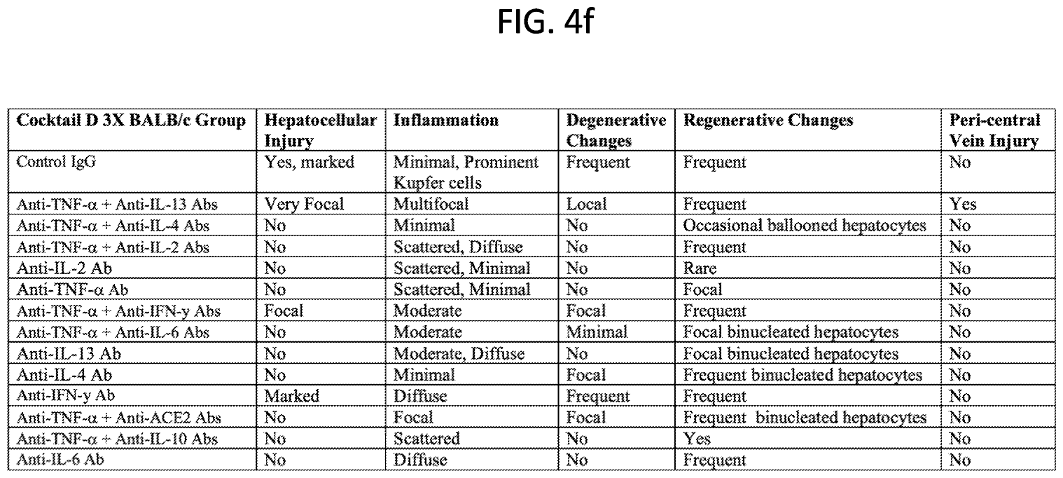

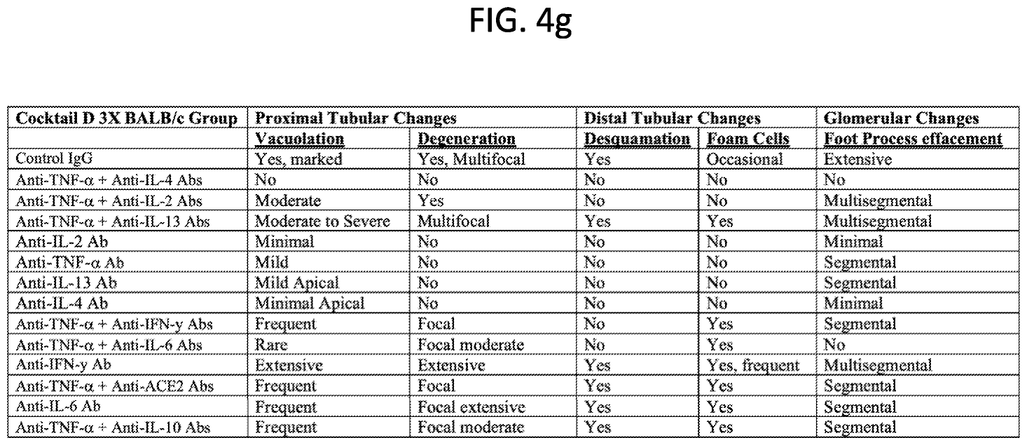

Injecting Cocktail D 3× in BALB/c mice caused high mortality at 24 hours (FIG. 4a) and was used to model the sick COVID-19 patient requiring Intensive Care. FIG. 4a-g shows possible therapeutic strategies for the effect of severe cytokine storms on systemic disease in BALB/c mice. Number of mice injected per group are shown in panel a. All depleting antibodies or control IgG were injected intravenously one hour after model induction. Large red asterisk indicates universal mortality. (FIG. 4a) Mortality table for BALB/c mice injected with Cocktail D 3× with control IgG or depleting antibodies. Since mortality was higher with metabolic cage use (5/6) than without (2/6) in the Control IgG group, timed urine collection for albuminuria was not conducted in these studies. (FIG. 4b) Serum cardiac Troponin I (cTPI3) levels on Day 1 among survivors of Cocktail D 3× dose injected mice, followed by control IgG or depleting antibodies. (FIG. 4c) Serum ALT activity levels on Day 1 among survivors of Cocktail D 3× dose injected mice, followed by control IgG or depleting antibodies. (FIG. 4d) Serum creatinine levels on Day 1 among survivors of Cocktail D 3× dose injected mice, followed by control IgG or depleting antibodies. (FIG. 4e) Morphometric comparison of cardiac histology between control and cytokine depletion groups. (FIG. 4f) Morphometric comparison of liver histology between control and cytokine depletion groups. (FIG. 4g) Morphometric comparison of kidney histology between control and cytokine depletion groups. Morphometric analysis n=3 mice per group. *P<0.05; P<0.01; ***P<0.001, all values based on two-tail analysis.

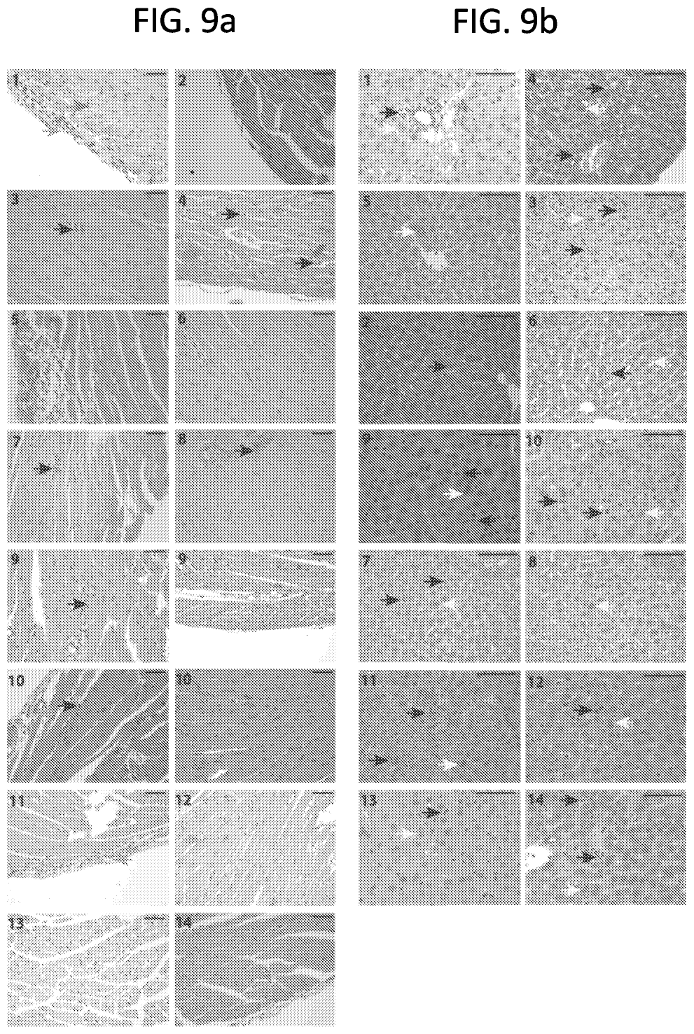

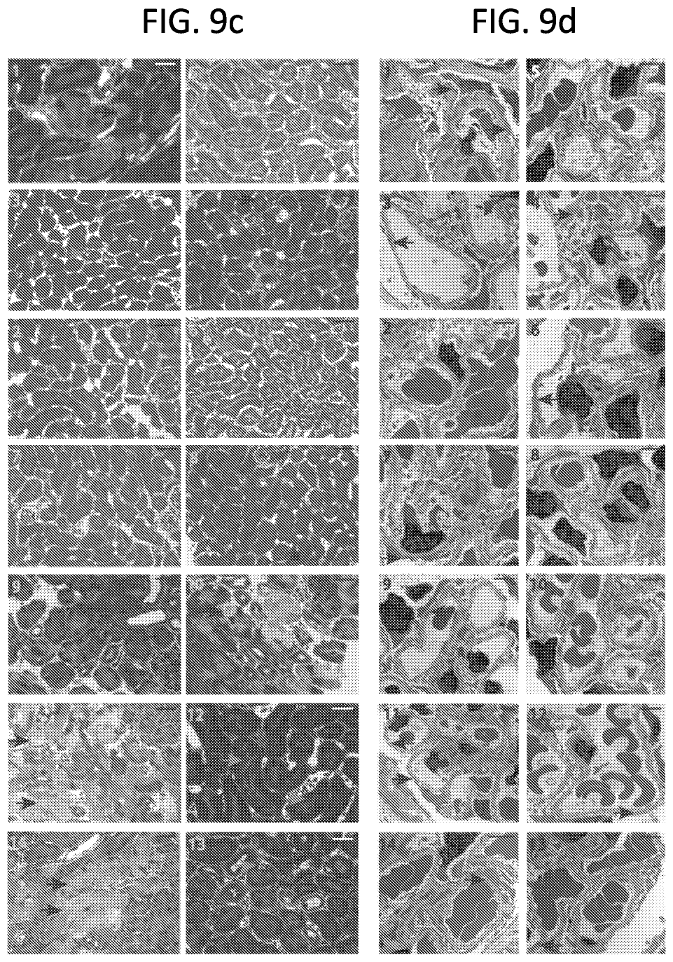

FIG. 9a-d shows histology for the severe injury model. Histological sections from studies of BALB/c mice (n=3 mice/group) euthanized 24 hours after Cocktail D dose 3× injection and additional antibodies or Control IgG injected one hour after model induction (see FIG. 4). The numbering code for each group is: “1”=Control IgG; “2”=Anti-IL-2 Ab; “3”=Anti-TNFα+Anti-IL-2 Abs; “4”=Anti-TNFα+Anti-IL-13 Abs; “5”=Anti-TNFα+Anti-IL-4-Abs; “6”=Anti-TNFα Ab; “7”=Anti-IL-13; “8”=Anti-IL-4 Ab; “9”=Anti-TNFα+Anti-IFNγ Abs; “10”=Anti-TNFα+Anti-IL-6 Abs; “11”=Anti-IFNγ Ab; “12”=Anti-TNFα+Anti-ACE2 Abs; “13”=Anti-TNFα+Anti-IL-10 Abs; “14”=Anti-IL-6 Ab. (FIG. 9a) Two columns of H&E-stained sections of the heart and pericardium. Myocytolysis (red arrows), inflammation (black arrows), hypereosinophilia (green arrows) and pericarditis (orange arrow) were noted. (FIG. 9b) Two columns of H&E-stained sections of the liver. Hepatocellular injury (red arrows), inflammation (black arrows), degenerative changes (green arrows), and regenerative changes (yellow arrows) were noted. (FIG. 9c) Two columns of Toluidine blue stained sections of the kidney showing gross tubular morphology. Tubular vacuolation (red arrows) and tubular degeneration (black arrows) were noted in proximal tubules. (FIG. 9d) Two columns of electron microscopy of the kidney showing images of glomeruli. Areas of podocyte foot process effacement (black arrows) were noted. Scale bars (a) 20 μm (b) 20 μm (c) 20 μm (d) 0.5 μm.

Metabolic cages for urine collection were not used in this study to avoid near universal mortality (5/6 dead) in the control IgG group. Depletion of TNF-α in combination with IL-2 or IL-13 or IL-4, or monotherapy for TNF-α, IL-13 or IL-4 depletion were most effective in disrupting cocktail component synergy, eliminated mortality and normalized overall activity in mice at 24 hours (FIG. 4a). These interventions, especially specific anti-TNF-α antibody-based combinations, were most efficacious in reducing serum levels of cTPI3, ALT and creatinine (FIG. 4b, c, d). Monotherapy for IL-2 depletion reduced organ injury effectively but mice still had some standing hair at 24 hours, indicating mild distress. Monotherapy for depletion of IL-6, IL-10 and IFN-γ were all counterproductive. Morphometric analysis of heart, liver and kidney showed significant improvement with the most efficacious regimens (FIG. 4e, f, g; FIG. 9a-d).

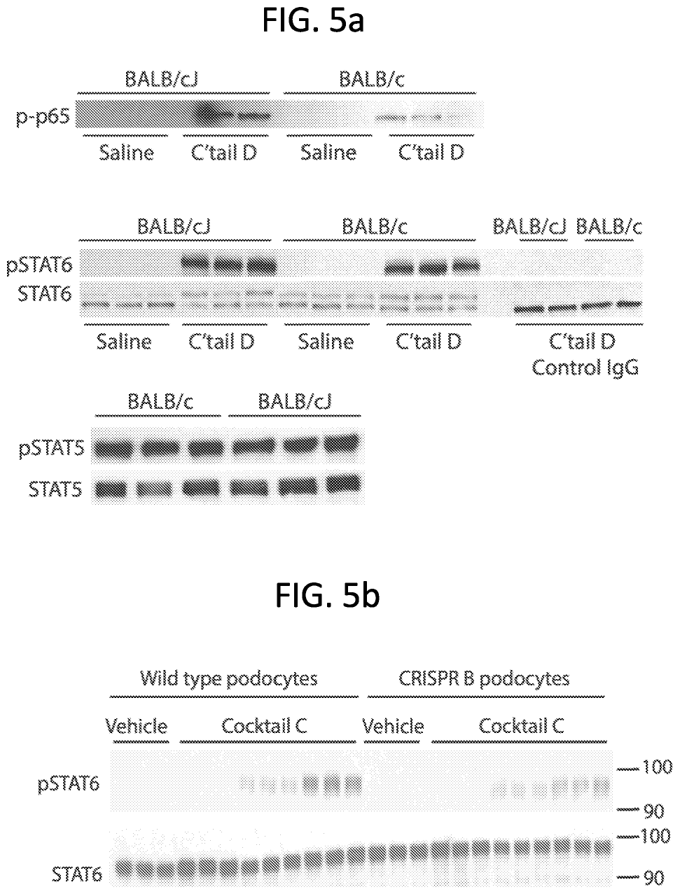

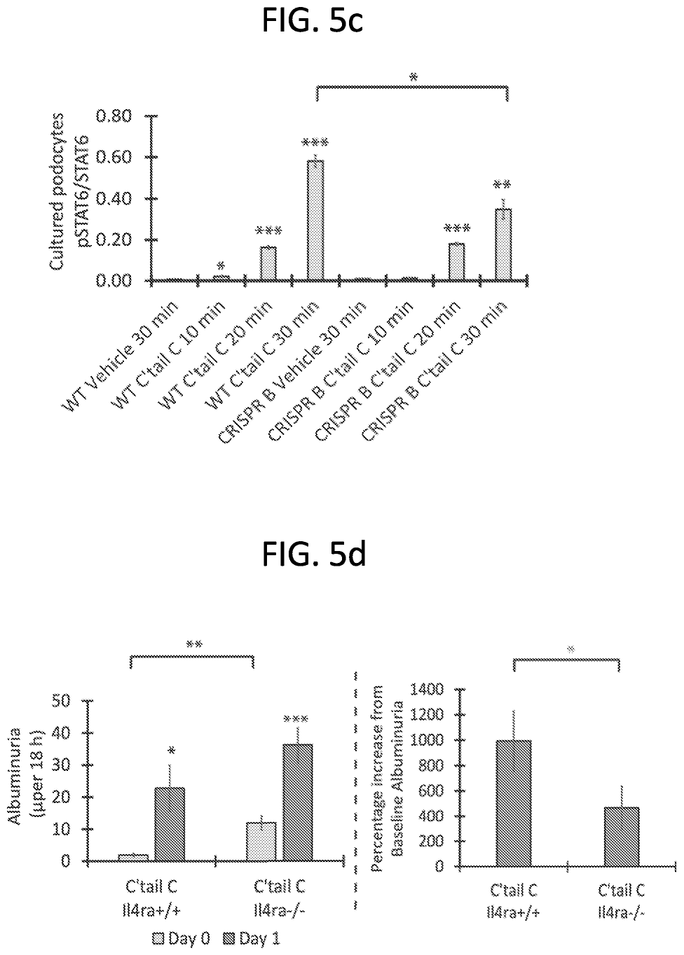

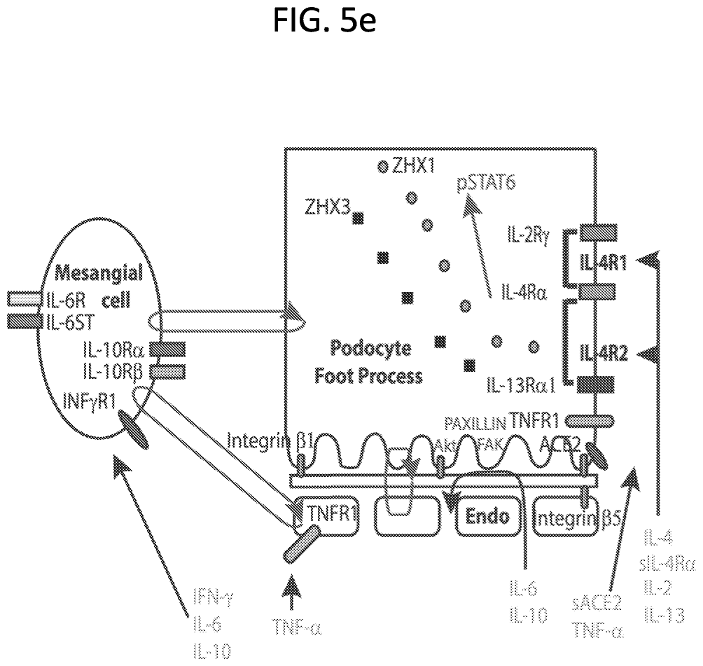

FIG. 5a-e shows the activation of signaling pathways by COVID cocktails and disease mechanisms. (FIG. 5a) Examples of NFκB/p-p65 (liver, 30 minutes, qualitative study), pSTAT6 (kidney 60 minutes) and pSTAT5 (heart, 15 minutes) activation by Western blot of whole organ protein extracts of mice (n=3 per group) injected with Cocktail D 3× or control saline. (FIG. 5b) Western blots to assess activation of pSTAT6 signaling in wild type and ZHX2 hypomorph (CRISPR B) cultured human podocytes incubated with human counterparts of Cocktail C (final concentration×/100,000; n=3 plates per condition). (FIG. 5c) Densitometry of Western blot of Cocktail C incubated wild type and CRISPR B podocytes from panel b. (FIG. 5d) Albuminuria in Il4ra^{-/-} and control BALB/cJ mice after injecting Cocktail C dose X/2 (left panel), and percentage increase in Day 1 albuminuria from baseline (right panel) (n=5 to 8 mice per group). (FIG. 5e) Schematic for potential binding of COVID cocktail components to specific receptors previously described in glomerular endothelial cell, mesangial cells and podocytes, and feedback loops (red) between these cells. *P<0.05; **P<0.01; ***P<0.001, all values based on two-tail analysis, except right panel in FIG. 5d is one-tail analysis.

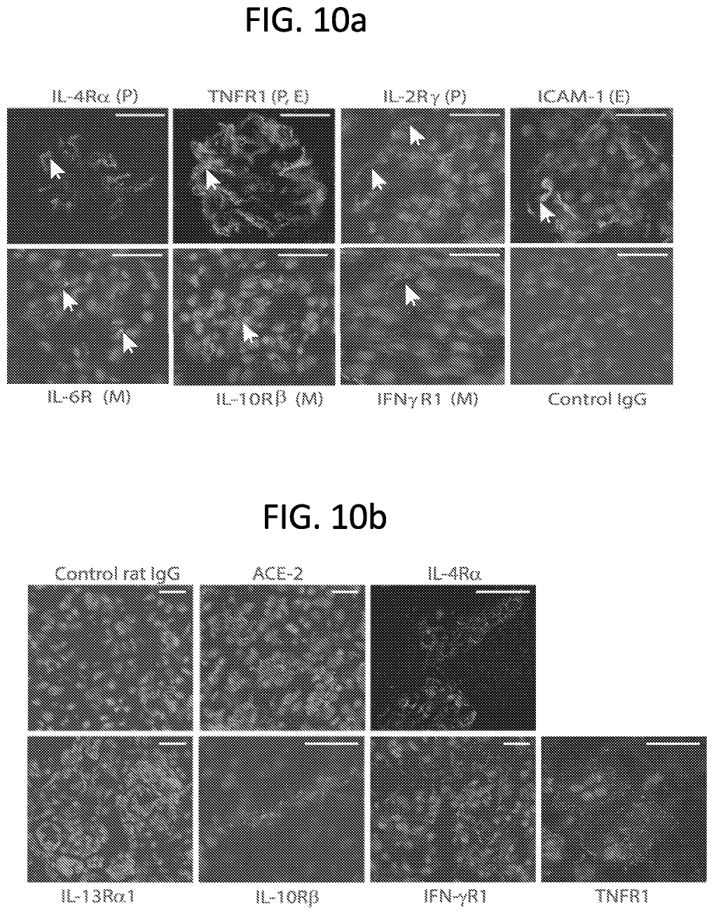

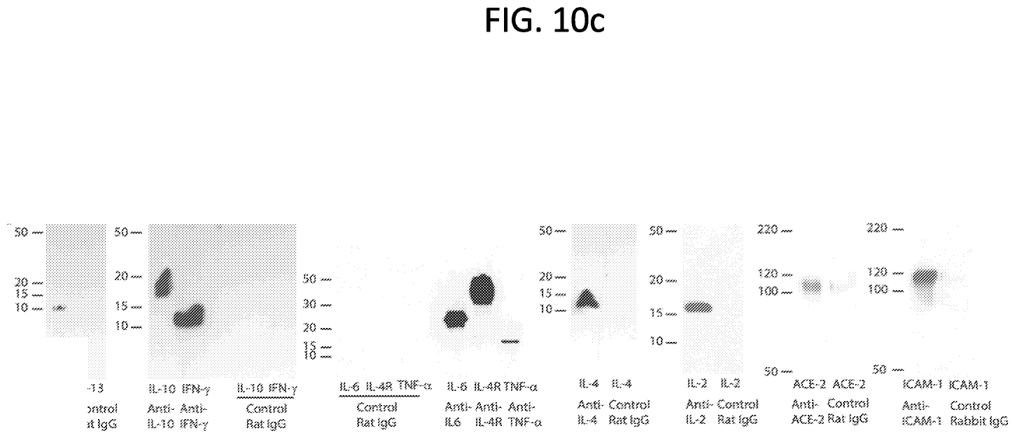

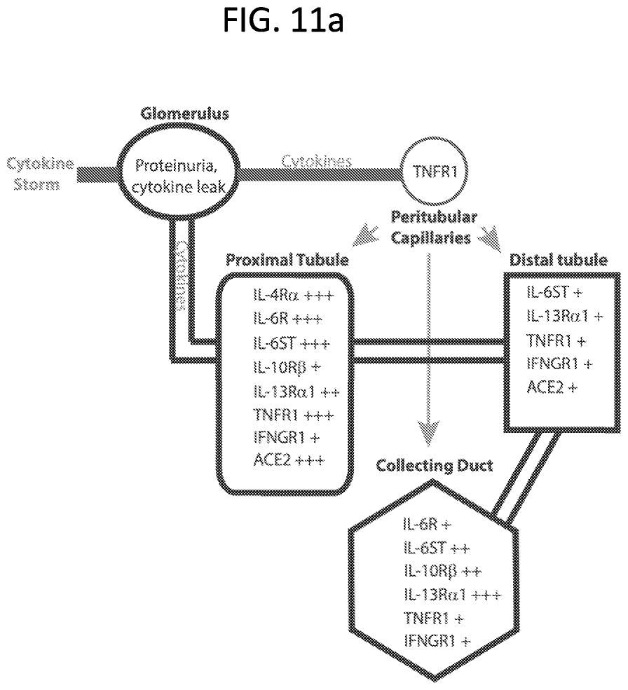

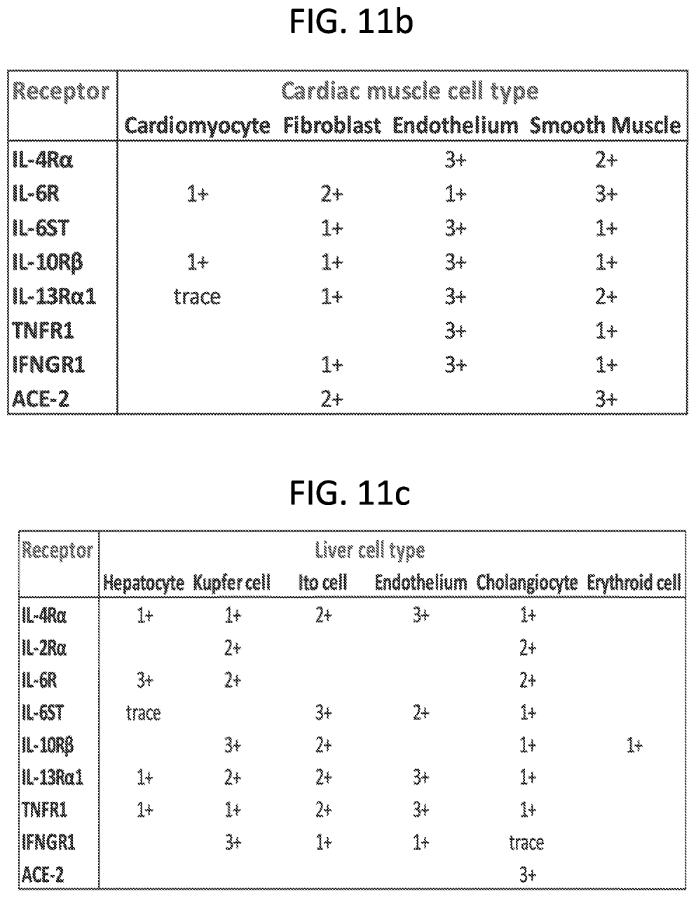

FIG. 10a-c: (FIG. 10a) Confocal expression of cytokine receptors in BALB/c mouse glomeruli. White arrows indicate receptor expression in podocytes (P), endothelial (E) and mesangial (M) cells. Since TNFR1 is expressed in podocytes and endothelial cells, only partial co-localization with nephrin (blue), a podocyte protein, is noted. Green color is nuclear stain. (FIG. 10b) Confocal expression (red) of ACE-2 and cytokine receptors in BALB/c mouse kidney tubules. Most images show proximal tubules, except IL-10Rβ image is collecting duct. (FIG. 10c) Western blot characterization of antibodies used for depletion studies using recombinant proteins that make up the cytokine cocktails. Scale bars (a) 20 μm (b) 20 μm.

### DISCUSSION

SARS-COV-2 infection starts in the respiratory tract, elicits a prominent immune response, and in some cases, involves other organs by direct infection as well. The magnitude of the extra-pulmonary involvement is often out of proportion to direct infection, suggesting that the innate and adaptive immune response to the primary infection may have a significant pathogenic role. This study focuses on the multisystem pathogenic effects of the extensive cytokine storm documented early in the pandemic.

Building de novo two viral cytokine storm models (SARS-COV-2/COVID-19) brings into focus the synergistic rather than the individual effects of components. Glomerular disease was used as the model of choice for mild cytokine storms, since it allowed us to compare the effects of two common viral infections on rare (e. g. relapse of MCD by a common cold) and common (COVID-19 induced proteinuria) clinical scenarios in the absence of other end organ damage.

When the severe COVID-19 cytokine storm was replicated in BALB/c mice using Cocktail D dose 1.8× or 3×, effects beyond glomerular injury were noted, including acute myocarditis, pericarditis, liver and kidney injury and significant acute all-cause mortality in the 3× dose injected mice. Since the cytokine storm origin was extrinsic to these organs, only mild to moderate inflammation, as also often noted in SARS-COV-2 infected patients, was present. Compared to BALB/c mice, Zhx2^{hypo/hypo} BALB/cJ mice developed less severe heart, liver and kidney injury and lower mortality, whereas the extent of glomerular injury was similar. As the literature suggests, the disparity between glomerular and other forms of injury is most likely related the predominantly cell membrane localization of ZHX proteins in podocytes, and largely nuclear expression in the heart, liver and kidney tubular cells.

The depleting antibodies were administered one hour after injection of the cytokine cocktail, which is sufficient time to initiate multi-pathway injury, since all mice injected with high dose Cocktail D were equally sick at the 6-hour time point. The improvement, or its lack, at 24 hours was reflective of the therapeutic efficacy of the depletion regimen.

The most effective regimens for severe cytokine storms in BALB/c mice, which would parallel most ICU admitted patients, were a combination depletion of TNFα with IL-2, IL-4 or IL-13. Using these combinations, there was no mortality, overall mouse activity normalized, and serum cTPI3, ALT and creatinine levels were closest to normal. Monotherapy for IL-2, TNFα, IL-4 and IL-13 depletion also eliminated mortality and overall activity was improved, but either biomarker levels tended to be higher than the combination groups, or mild distress e. g. standing hair, persisted. Monotherapy for depletion of IL-6 or IL-10 fared worse than other groups. The intermediate dose of Cocktail D 1.8× was also studied in BALB/c mice to mimic the inpatient non-intensive care setting, since there was no mortality and less multiorgan injury. These mice responded well to select single cytokine depletion. In mild Cocktail C models (Dose X/2), anti-TNF-α, anti-IL-10, anti-IFN-γ, and select anti-TNFα antibody-based combinations were effective in reducing albuminuria.

The concept of synergy between different cytokinesin cytokine cocktails and not by comparable or higher doses of individual cytokines is clearly illustrated. Cytokine depletion regimens described in this study likely exert their effects by reducing this synergy.

At least two of likely numerous pathways active in podocytes during cytokine storms were defined. Migration of ZHX proteins from the cell membrane and pSTAT6 signaling were activated downstream of IL-4Rα by COVID-19 cocktails. In addition, the pathogenic effects of circulating sACE-2 in COVID-19 cocktails could be mediated via interaction with integrins⁴¹. The increase in plasma sIL-4Rα levels in COVID-19 patients with proteinuria suggests that this pathway is likely to be active in this subset of patients.

As will be appreciated from the descriptions herein, a wide variety of aspects and embodiments are contemplated by the present disclosure, examples of which include, without limitation, the aspects and embodiments listed below:
Methods of inhibiting, treating, or preventing the effects of cytokine storms as the result of viral infections in patients comprising inhibiting, neutralizing or depleting one or more cytokines from the patient;
Methods of depleting two or more cytokines in order to reduce the mortality caused by severe cytokine storms;
Methods of treating the effects of acute heart injury, acute liver injury and acute kidney injury used by cytokine storms; the inventors contemplate that the cytokine storms can be caused by viral infections in some embodiments;
Methods of reducing mortality caused by cytokine storms; the inventors contemplate that the cytokine storms can be caused by viral infections in some embodiments;
Methods to prevent multi-organ injury induced by a cytokine storm comprising the inhibition, neutralization, or depletion more than one cytokine;
Methods for treating or preventing the effects of post-acute sequelae of a SARS-Cov-2 infection comprising the inhibition, neutralization or depletion of one or more cytokines;
Methods for preventing the relapse of a viral infection where the methods involve providing treatments that inhibit, neutralize or deplete one or more cytokines;
Methods for treating or preventing the effects of SARS-COV-2 virus mRNA vaccines comprising the inhibition, neutralization or depletion of one or more cytokines;
Animal models for cytokine storms induced by viral infections and other disease states to test methods of treating or preventing the effects of said cytokine storm; and,
Methods are for treating a viral infection.

Methods of treating a cytokine storm that is not of viral origin including bacterial, fungal or parasitic infections, cancer, organ transplantation, or results from the change in the systemic cytokine milieu of a multisystem disease like diabetes mellitus or metabolic syndrome.

Methods of inhibiting, neutralizing or depleting one or more cytokines by the administration of an agent to the patient where the agent comprises an adeno-associated virus (AAV) or lentovirus-containing an a short-hairpin RNA (shRNA) against one or more cytokines. The shRNA can be made or is commercially available and can be attached to or part of any vector known in the art including plasmids, viral vectors, bacteriophages, cosmids, and artificial chromosomes.

Methods of depleting one or more cytokines is by the administration of a monoclonal or polyclonal antibody directed against the one or more cytokines. In yet other embodiments, the agent comprises a monoclonal or polyclonal antibody directed against one or more cytokines.

Methods of depleting one or more cytokines is by the administration of an siRNA or antisense oligonucleotide that targets one or more cytokines.

Methods of depleting one or more cytokines is by the administration of an antagonist that binds to a cytokine-mediated receptor and prevents the binding of one or more cytokines.

In any of the disclosed methods, the one or more cytokines to be inhibited, neutralized or depleted comprise TNFα, IL-2, IL-4, IL-13, IFN-γ or IL-6. It will be understood for the disclosure herein that depending upon the severity of the viral infection or other condition being treated, the inhibition, neutralization or depletion more than one cytokine may be more effective that depletion of a single cytokine.

While embodiments of the present disclosure have been described herein, it is to be understood by those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-53. (canceled)

54. A method of inhibiting, treating or preventing the effects of a mild to moderate cytokine storm in a patient in need thereof, comprising administering an inhibitor of one or more cytokines to the patient, wherein the inhibitor of one or more cytokines neutralizes or depletes the cytokine or its effect from the patient, resulting in an alleviation of the symptoms of the mild cytokine storm.

55. The method of claim 54, wherein the cytokine storm is the result of a viral infection.

56. The method of claim 55, wherein the viral infection is caused by an infection selected from the group consisting of: SARS-COV-1, SARS-COV-2, influenza, parainfluenza, Respiratory Syncytial Virus (RSV), adenoviruses, enteroviruses, other coronaviruses, cytomegalovirus (CMV), Epstein Barr Virus (EBV), Middle East Respiratory Syndrome (MERS), and Ebola virus.

57. The method of claim 54, wherein the mild to moderate cytokine storm is the result of a disease state selected from the group consisting of: fungal infections, parasitic infections, cancer, organ transplantation, diabetes mellitus, and metabolic syndrome.

58. The method of claim 54, wherein the inhibitor is an antibody or an antibody fragment directed against the one or more cytokines, selected from the group consisting of a polyclonal antibody, a monoclonal antibody, a bivalent antibody, and an antibody fragment of any of the foregoing.

59. The method of claim 58, wherein said antibody or antibody fragment is bivalent and targets two different cytokines.

60. The method of claim 54, wherein the effects of the cytokine storm are non-respiratory effects.

61. The method of claim 60, wherein the non-respiratory effects are selected from acute heart injury, acute liver injury, and acute kidney injury.

62. The method of claim 54, wherein the one or more cytokines comprise TNFα, IL-2, IL-4, IL-13, IFN-γ, or a combination of two or more of the foregoing.

63. A method of inhibiting, treating or preventing the effects of a severe cytokine storm in a patient in need thereof, comprising administering inhibitors of two or more cytokines to the patient, wherein the inhibitors neutralize or deplete said cytokines or their effects from the patient, resulting in an alleviation of the symptoms of the severe cytokine storm.

64. The method of claim 63, wherein the cytokine storm is the result of a viral infection.

65. The method of claim 64, wherein the viral infection is caused by an infection selected from the group consisting of: SARS-COV-1, SARS-COV-2, influenza, parainfluenza, Respiratory Syncytial Virus (RSV), adenoviruses, enteroviruses, other coronaviruses, cytomegalovirus (CMV), Epstein Barr Virus (EBV), Middle East Respiratory Syndrome (MERS), and Ebola virus.

66. The method of claim 63, wherein the severe cytokine storm is the result of a disease state selected from the group consisting of bacterial infections, fungal infections, parasitic infections, cancer, organ transplantation, diabetes mellitus, and metabolic syndrome.

67. The method of claim 63, wherein the inhibitor is an antibody or an antibody fragment directed against the one or more cytokines, selected from the group consisting of a polyclonal antibody, a monoclonal antibody, a bivalent antibody, and an antibody fragment of any of the foregoing.

68. The method of claim 67, wherein said antibody or antibody fragment is bivalent and targets two different cytokines.

69. The method of claim 63, wherein the effects of the cytokine storm are non-respiratory effects.

70. The method of claim 69, wherein the non-respiratory effects are selected from acute heart injury, acute liver injury, and acute kidney injury.

71. The method of claim 63, wherein the two or more cytokines comprise TNFα, IL-2, IL-4, IL-13, IFN-γ, or IL-6.

72. A method of treating or preventing a side effect of a vaccine in a patient, wherein the vaccine is a SARS-COV-2 mRNA vaccine, the method comprising: administering an inhibitor of a cytokine to the patient, wherein the inhibitor depletes or neutralizes the cytokine.

73. The method of claim 72, wherein the inhibitor inhibits a second cytokine, and wherein the inhibitor depletes or neutralizes the second cytokine.


SEQUENCE LISTING (32 sequences)

SEQ ID NO: 1 - DNA, 25 bp, synthetic construct
  source 1..25: mol_type = other DNA
caccgactggtaaaccacttagggc

SEQ ID NO: 2 - DNA, 25 bp, synthetic construct
  source 1..25: mol_type = other DNA
aaacgccctaagtggtttaccagtc

SEQ ID NO: 3 - DNA, 28 bp, synthetic construct
  source 1..28: mol_type = other DNA
gcatatacgatacaaggctgttagagag

SEQ ID NO: 4 - DNA, 36 bp, synthetic construct
  source 1..36: mol_type = other DNA
cgggccggatccctagatgtagcattaccagggtgg

SEQ ID NO: 5 - DNA, 41 bp, synthetic construct
  source 1..41: mol_type = other DNA
ggccgaagcttgcagagaagatcacgatagattagaagatg

SEQ ID NO: 6 - DNA, 22 bp, synthetic construct
  source 1..22: mol_type = other DNA
ggtttccttgttatatcaccag

SEQ ID NO: 7 - DNA, 64 bp, synthetic construct
  source 1..64: mol_type = other DNA
gctctaggatgactggtaaaccacttagggcagtcgtccccagacctggtctgtggcctgttag

SEQ ID NO: 8 - DNA, 64 bp, synthetic construct
  source 1..64: mol_type = other DNA
ctaacaggccacagaccaggtctggggacgactgccctaagtggtttaccagtcatcctagagc

SEQ ID NO: 9 - DNA, 45 bp, synthetic construct
  source 1..45: mol_type = other DNA
gattatctttctagggttaacgaacttcaagtaatcaagagcagc

SEQ ID NO: 10 - DNA, 43 bp, synthetic construct
  source 1..43: mol_type = other DNA
cgcagactatctttctagggttaactttgtagaatgcttctcg

SEQ ID NO: 11 - DNA, 43 bp, synthetic construct
  source 1..43: mol_type = other DNA
cgagaagcattctacaaagttaaccctagaaagatagtctgcg

SEQ ID NO: 12 - DNA, 45 bp, synthetic construct
  source 1..45: mol_type = other DNA
gctgctcttgattacttgaagttcgttaaccctagaaagataatc

SEQ ID NO: 13 - DNA, 44 bp, synthetic construct
  source 1..44: mol_type = other DNA
acactgacgacatggttctacagtctctgaaacatagaaggcac

SEQ ID NO: 14 - DNA, 45 bp, synthetic construct
  source 1..45: mol_type = other DNA
tacggtagcagagacttggtctgagaatctaataccgctgatctg

SEQ ID NO: 15 - DNA, 25 bp, synthetic construct
  source 1..25: mol_type = other DNA
caccgacccatccatacacttaccc

SEQ ID NO: 16 - DNA, 25 bp, synthetic construct
  source 1..25: mol_type = other DNA
aaacgggtaagtgtatggatgggtc

SEQ ID NO: 17 - DNA, 28 bp, synthetic construct
  source 1..28: mol_type = other DNA
gcatatacgatacaaggctgttagagag

SEQ ID NO: 18 - DNA, 46 bp, synthetic construct
  source 1..46: mol_type = other DNA
ggcggcactagtctagctggcttgactttacaagacgattccatcc

SEQ ID NO: 19 - DNA, 40 bp, synthetic construct
  source 1..40: mol_type = other DNA
gggcggatccctgcactcagtattctgcaagtcctgtagc

SEQ ID NO: 20 - DNA, 18 bp, synthetic construct
  source 1..18: mol_type = other DNA
cgatctcctgacctcaag

SEQ ID NO: 21 - DNA, 167 bp, synthetic construct
  source 1..167: mol_type = other DNA
gtgcctggcctgttatgatcttcttactcatttgatagcaccagtgtcctgagaaaaataacatatactccattacccatccatacacttacccaggcactcattcaccatattaactagatagacacatgatgttgctgctcctgttgatgataacaatgttgagg

SEQ ID NO: 22 - DNA, 166 bp, synthetic construct
  source 1..166: mol_type = other DNA
cctcaacattgttatcatcaacaggagcagcaacatcatgtgtctatctagttaatatggtgaatgagtgactgggtaagtgtatggatgggtaatggagtatatgttatttttctcaggacactggtgctatcaaatgagtaagaagatcataacaggccaggac

SEQ ID NO: 23 - DNA, 59 bp, synthetic construct
  source 1..59: mol_type = other DNA
cgtcacaatatgattatctttctagggttaactagatagacacatgatgttgctgctcc

SEQ ID NO: 24 - DNA, 56 bp, synthetic construct
  source 1..56: mol_type = other DNA
cgtcaattttacgcagactatctttctagggttaatatggtgaatgagtgactggg

SEQ ID NO: 25 - DNA, 56 bp, synthetic construct
  source 1..56: mol_type = other DNA
cccagtcactcattcaccatattaaccctagaaagatagtctgcgtaaaattgacg

SEQ ID NO: 26 - DNA, 59 bp, synthetic construct
  source 1..59: mol_type = other DNA
ggagcagcaacatcatgtgtctatctagttaaccctagaaagataatcatattgtgacg

SEQ ID NO: 27 - DNA, 60 bp, synthetic construct
  source 1..60: mol_type = other DNA
acactgacgacatggttctacagttatgatcttcttactcatttgatagcaccagtgtcc

SEQ ID NO: 28 - DNA, 52 bp, synthetic construct
  source 1..52: mol_type = other DNA
tacggtagcagagacttggtctgaaaggagcagtgttgatctagagagagcc

SEQ ID NO: 29 - DNA, 21 bp, synthetic construct
  source 1..21: mol_type = other DNA
cggaactggctgaatcagact

SEQ ID NO: 30 - DNA, 24 bp, synthetic construct
  source 1..24: mol_type = other DNA
cagcacagcagttctaacagactt

SEQ ID NO: 31 - DNA, 15 bp, synthetic construct
  source 1..15: mol_type = other DNA
tgcagaggctggcca

SEQ ID NO: 32 - DNA, 10 bp, synthetic construct
  source 1..10: mol_type = other DNA
cacacacaca